(19) United States
(12) Patent Application Publication
(10) Pub. No.: US 2024/0002436 A1
(43) Pub. Date: Jan. 4, 2024

(54) RESPIRATORY TREATMENTS

(71) Applicant: Hofseth Biocare ASA, Alesund (NO)

(72) Inventors: Bomi FRAMROZE, Portola Valley, CA (US); Crawford Linden Alexander CURRIE, London (GB)

(73) Assignee: Hofseth Biocare ASA, Alesund (NO)

(21) Appl. No.: 18/036,382

(22) PCT Filed: Nov. 15, 2021
(86) PCT No.: PCT/US2021/059416

§ 371 Date: May 10, 2023

Related U.S. Application Data
(60) Provisional application No. 63/211,972, filed on Jun. 17, 2021.
(60) Provisional application No. 63/114,976, filed on Nov. 17, 2020.

(51) Int. Cl. C07K 5/103; C07K 5/107; C07K 5/062; C07K 5/065

(52) U.S. Cl. CPC C07K 5/101; C07K 5/1016; C07K 5/1008; C07K 5/06026; C07K 5/06034; C07K 5/06078; A61K 38/00

(57) ABSTRACT

Provided herein are methods for treating certain inflammatory and/or respiratory conditions, disorders and diseases in humans using compositions comprising lipopeptides, including microcolins. The compositions are orally administered. The humans suitable for such treatments include those largely resistant to medical and surgical interventions, such as steroid treatments. The compositions described herein have also been found to reduce cosinophil effector function.

### CROSS-REFERENCE TO RELATED APPLICATIONS

This application claims priority to U.S. Provisional Patent Application Nos. 63/114,976, filed Nov. 17, 2020, and 63/211,972, filed Jun. 17, 2021, each of which is incorporated herein by reference in its entirety.

### FIELD

The present disclosure relates generally to respiratory treatments, and more specifically to the use of lipopeptides, including microcolins and analogues thereof, to treat respiratory conditions, diseases or disorders, such as asthma, in a subgroup of patients that exhibit resistance to steroid therapies.

### BACKGROUND

Inflammation is an immunological conundrum. On the one hand, the physiological changes that accompany inflammation allow us to mount an acute response to external threats that would otherwise have wiped out the human species. On the other, chronic inflammation, where age or external stressors keep our immune system in overdrive, can contribute to many debilitating diseases ranging from Alzheimer's to diabetes and bronchial asthma.

An eosinophil is a type of white blood cell stored in tissues throughout the body and continually replenished from the bone marrow. Eosinophils typically have a two-day lifespan in blood, but inflammatory conditions such as infections and allergic diseases will extend the lifespan up to two weeks by eosinophil-activating cytokines. See Park Y M & Bochner B S, Allergy Asthma Immunol Res. 2010, 2:87-101. An eosinophil count is a blood test that measures the quantity of eosinophils in the human body. Elevated levels, usually measured during routine complete blood count testing, indicate an infection or allergy.

Activated eosinophils, which are promoted by eosinophil-activating cytokines under inflammatory conditions, are a major source of reactive oxygen species, cytotoxic proteins and proinflammatory cytokines. They signal the activation of resident tissue cells such as epithelial, endothelial and fibroblast cells, leading to the progression of inflammation and mucus secretion. Eosinophils are therefore potent activators and modulators of diseases such as bronchial asthma, atopic dermatitis' and colitis ulcerosa. See Hogan S P, Int Arch Allergy Immunol. 2007, 143(Suppl 1):3-14; Simon D et al., Allergy. 2004, 59:561-570; Wedemeyer J & Vosskuhle K., Best Pract Res Clin Gastroenterol. 2008, 22:537-549. Further, in asthmatics, levels of eosinophil granule proteins such as eosinophil cationic protein (ECP) and eosinophil peroxidase (EPO) largely correlate with asthma severity. See Parra A, et al., J Investig Allergol Clin Immunol. 1999; 9:27-34. Eosinophilic inflammation of the upper airways may also occur independent of allergy, as observed in chronic rhinosinusitis (CRS) subjects. See Hutcheson P S, et al., J Rhinol Allergy. 2010, 24:405-408. Such individuals represent a unique subgroup who are largely resistant to medical and surgical interventions and who could show immediate benefit by therapy that targets eosinophilic expansion and effector functions.

### BRIEF SUMMARY

In some aspects, provided herein respiratory treatments, such as asthma treatments, using lipopeptides. In some embodiments, the lipopeptides are microcolins, and structural analogues thereof. In some variations, microcolin A or microcolin B, or a combination thereof, is administered in the respiratory treatments.

In some embodiments, the asthma is bronchial asthma. In certain embodiments, the treatments provided target a subgroup of patients (e.g., asthma patients) that are largely resistant to medical and surgical interventions, including steroid therapies.

In certain aspects, provided is a method for treating an inflammatory condition, disorder or disease in a human in need thereof, comprising: orally administering to the human an effective dose of a composition comprising at least one lipopeptide, including for example, at least one microcolin, or structural analogues thereof, to treat the inflammatory condition, disorder or disease.

In certain aspects, provided is a method for treating a respiratory condition, disorder or disease in a human in need thereof, comprising: orally administering to the human an effective dose a composition comprising at least one lipopeptide, including for example, at least one microcolin, or structural analogues thereof, to treat the respiratory condition, disorder or disease.

In certain aspects, provided is a method for reducing eosinophil effector function in a human in need thereof, comprising administering to the human a composition comprising at least one lipopeptide, including for example, at least one microcolin, or structural analogues thereof, to reduce eosinophil effector function.

In other aspects, provided is an article of manufacture, comprising: a container comprising a composition comprising at least one lipopeptide, including for example, at least one microcolin, or structural analogues thereof; and a label containing instructions for use of such composition.

In yet other aspects, provided is a kit, comprising: a dosage form of a composition comprising at least one lipopeptide, including for example, at least one microcolin, or structural analogues thereof; and a package insert containing instructions for use of such composition.

In some variations of the foregoing aspects, the dosage form is a syrup, chewable, capsule or soft gel. In other variations, the compositions provided herein are formulated for aerosol delivery.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In some aspects, provided herein are methods for treating inflammatory conditions, disorders or diseases in humans in need thereof by orally administering a composition comprising at least one lipopeptide.

In some embodiments, the lipopeptide is microcolin. In one embodiment, the lipopeptide is microcolin A. In other embodiments, the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-hydroxy-2-pyrrolidinyl)carbonyl]-5-methyl-3-pyrrolin-2-one.

In some embodiments, the lipopeptide is a microcolin analogue. In one embodiment, the lipopeptide is an analogue of microcolin A or analogue of microcolin B. In other embodiments, the lipopeptide is a branched alkyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one. In certain variations, the branched alkyl is branched at the 1- and 3-positions of the alkyl chain, referring to the following positions on the exemplary alkyl chain depicted below:

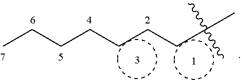

In yet other embodiments, the lipopeptide is a branched alkyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-N-[pyrrole-2,5-dione].

In other embodiments, the lipopeptide is alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-N-[pyrrole-2,5-dione], or alkenyl-[N-methyl aminoacid with hydrophobic side chain]-[aminoacid with polar uncharged sidechain]-[N-methyl aminoacid with hydrophobic side chain]-N-[pyrrolidine-2-carbonyl]-N-[pyrrole-2,5-dione].

In one variation, the lipopeptide is alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-N-[pyrrole-2,5-dione], or alkenyl-[N-methyl aminoacid with hydrophobic side chain]-[aminoacid with polar uncharged sidechain]-[N-methyl aminoacid with hydrophobic side chain]-N-[pyrrolidine-2-carbonyl]-N-[pyrrole-2,5-dione]. In one variation, the lipopeptide is alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-1H-[pyrrole-2,5-dione], or alkenyl-[N-methyl aminoacid with hydrophobic side chain]-[aminoacid with polar uncharged sidechain]-[N-methyl aminoacid with hydrophobic side chain]-N-[pyrrolidine-2-carbonyl]-1H-[pyrrole-2,5-dione].

### Compounds of Formula (I)

In certain embodiments, the composition administered comprises at least one compound of formula (I):

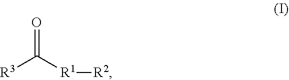

a salt thereof, including any isomers of the foregoing, wherein:

R¹ is at least one optionally substituted amino acid moiety;

R² is

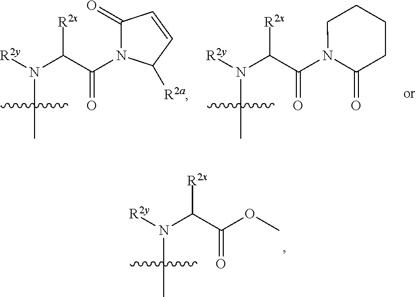

wherein

R^{2a} is H, oxo or optionally substituted alkyl;

R^{2x} is alkyl, optionally substituted with —OH or —COOH;

R^{2y} is H or alkyl; or

R^{2x} and R^{2y} are taken together with the atoms to which they are attached to form an optionally substituted 5-membered heterocycle; and

R³ is alkyl.

In some variations of formula (I):

R¹ is at least one optionally substituted amino acid moiety;

R² is

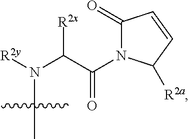

wherein:

R^{2a} is H or optionally substituted alkyl;

R^{2x} is alkyl, optionally substituted with —OH or —COOH;

R^{2y} is H or alkyl; or

R^{2x} and R^{2y} are taken together with the atoms to which they are attached to form an optionally substituted 5-membered heterocycle; and

R³ is alkyl.

In some variations, R¹ is a sequence of three optionally substituted amino acid moieties. In certain variations, R¹ is —R^{1a}—R^{1b}—R^{1c}—, wherein R^{1a} is an optionally substituted amino acid moiety with a hydrophobic side chain; R^{1b} is an optionally substituted amino acid moiety with a polar uncharged side chain; and R^{1c} is an optionally substituted amino acid moiety with a hydrophobic side chain. In certain variations, R^{1a} is an optionally substituted amino acid moiety selected from the group consisting of Leu, Val, and Gly; R^{1b} is an optionally substituted amino acid moiety selected from the group consisting of Thr, Ser, and Cys; and R^{1c} is an optionally substituted amino acid moiety selected from the group consisting of Val, Leu, and Gly. In certain variations, R^{1a} is an optionally substituted amino acid moiety selected from the group consisting of Leu, Val, and Gly; R^{1b} is a substituted amino acid moiety selected from the group consisting of Thr, Ser, and Cys, substituted with an acetyl group (—C(O)CH₃) through the oxygen atom of the amino acid moiety; and R^{1c} is an optionally substituted amino acid moiety selected from the group consisting of Val, Leu, and Gly.

In some variations, R¹ is Leu-OAcThr-Val, wherein each Leu and Val is N-alkylated; Val-OAcThr-Leu, wherein each Val and Leu is N-alkylated; Gly-OAcThr-Gly, wherein each Gly is N-alkylated; Leu-OAcSer-Val, wherein each Leu and Val is N-alkylated; or Leu-SAcCys-Val, wherein each Leu and Val is N-alkylated. In one variation, R¹ is Leu-OAcThr-Val, wherein each Leu and Val is N-methylated; Val-OAcThr-Leu, wherein each Val and Leu is N-methylated; Gly-OAcThr-Gly, wherein each Gly is N-methylated; Leu-OAcSer-Val, wherein each Leu and Val is N-methylated; or Leu-SAcCys-Val, wherein each Leu and Val is N-methylated.

In other variations, R¹ is —R^{1a}—R^{1b}—R^{1c}—, wherein R^{1a} is an optionally substituted amino acid moiety selected from the group consisting of Leu, and Phe; R^{1b} is an optionally substituted Thr; and R^{1c} is an optionally substituted amino Val.

In certain variations, R¹ is Leu-Thr-Val, wherein each Leu and Val is N-methylated; Leu-OAcThr-Val, wherein each Leu and Val is N-methylated; Phe-OAcThr-Val, wherein each Phe and Val is N-methylated; and Phe-Thr-Val, wherein each Phe and Val is N-methylated.

It should be understood that in the foregoing variations, “OAc-amino acid moiety” refers to the amino acid moiety substituted with an acetyl group via the oxygen atom of the amino acid moiety (as the case may be). For example, “OAcThr” or “OAcSer” refers to threonine or serine, respectively, substituted with an acetyl group through the oxygen atom of the amino acid side chain. Similarly, it should be understood that in the foregoing variations, “SAc-amino acid moiety” refers to the amino acid moiety substituted with an acetyl group via the sulfur atom of the amino acid moiety (as the case may be). For example, “SAcCys” refers to cysteine substituted with an acetyl group through the sulfur atom of the amino acid side chain.

The descriptions herein refer to the following abbreviations: “Leu” refers to leucine; “Val” refers to valine, “Gly” refers to glycine; “Thr” refers to threonine; “Ser” refers to serine; “Cys” refers to cysteine; “Ala” refers to alanine; “Ile” refers to isoleucine; “Met” refers to methionine; “Phe” refers to phenylalanine; “Tyr” refers to tyrosine; “Trp” refers to tryptophan. It should further be understood that, in some variations where an amino acid is “N-alkylated”, the nitrogen of the amino acid backbone is alkylated. Similarly, in some variations where an amino acid is “N-methylated”, the nitrogen of the amino acid backbone is alkylated with a methyl group.

In some variations, R^{2a} is H. In other variations, R^{2a} is alkyl. In certain variations, R^{2a} is C₁₋₁₀ alkyl, or C₁₋₅ alkyl, or C₁₋₃ alkyl. In one variation, R^{2a} is methyl.

In some variations, R^{2x} is H. In other variations, R^{2x} is optionally substituted alkyl. In certain variations, the alkyl is C₁₋₂₀ alkyl, C₁₋₁₅ alkyl, C₁₋₁₀ alkyl, or C₁₋₅ alkyl, or C₁₋₃ alkyl. In certain variations, the alkyl is substituted with —OH. In yet other variations, the alkyl is substituted with —COOH.

In some variations of the foregoing, R^{2y} is H. In other variations, R^{2y} is alkyl. In certain variations, the alkyl is C₁₋₂₀ alkyl, C₁₋₁₅ alkyl, C₁₋₁₀ alkyl, or C₁₋₅ alkyl, or C₁₋₃ alkyl, or methyl.

In some variations, R^{2x} and R^{2y} are taken together with the atoms to which they are attached to form an optionally substituted 5-membered heterocycle. In certain variations, the heterocycle is pyrrolidine. In certain variations, the heterocycle is unsubstituted, or substituted with one or more of —OH or —COOH.

In some variations, R² is

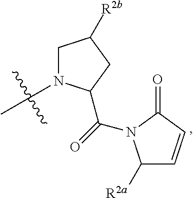

wherein R^{2b} is H or OH. In one variation, R² is

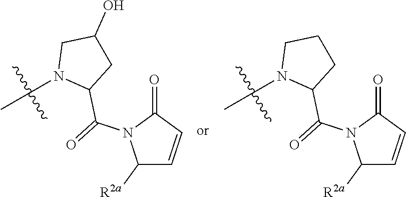

In certain variations, R² is:

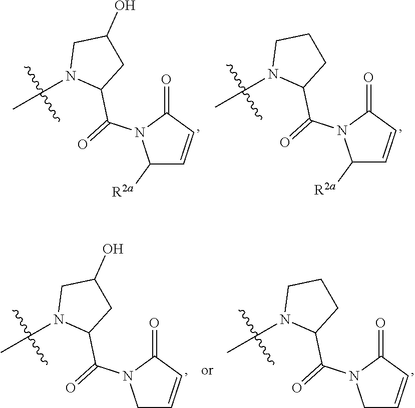

In certain variations, R² is:

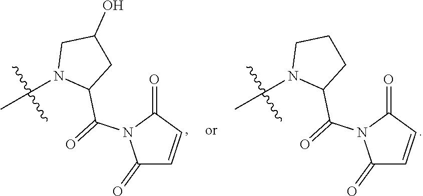 [SMILES: *N1CC(O)CC1C(=O)N1C(=O)C=CC1=O ; *N1CCCC1C(=O)N1C(=O)C=CC1=O]

In some variations, R² is:

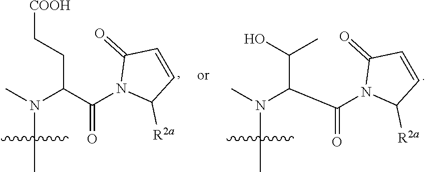

In certain variations, R² is:

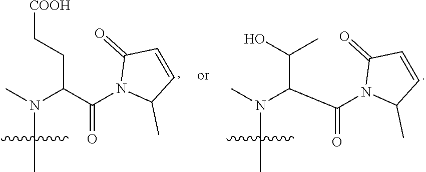 [SMILES: *N(C)C(CCC(=O)O)C(=O)N1C(=O)C=CC1C ; *N(C)C(C(=O)N1C(=O)C=CC1C)C(C)O]

In some variations, R³ is a branched alkyl. In other variations, R³ is C₁₋₂₀ alkyl, C₁₋₁₀ alkyl, or C₅₋₁₀ alkyl, or C₇₋₉ alkyl. In certain variations of the foregoing, the alkyl is unbranched or branched. In one variation, the alkyl is branched at the 1- and/or 3-position of the alkyl chain. In some variations, R³ is:

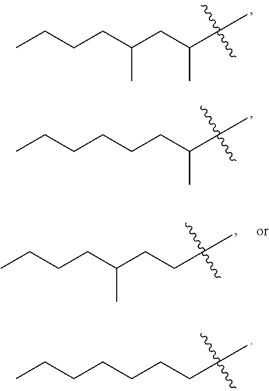

### Compound of Formula (II)

In some embodiments, the composition administered comprises: at least one compound of formula (II):

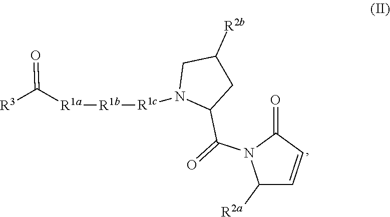

a salt thereof, including any isomers of the foregoing, wherein:
R^{1a} is an optionally substituted amino acid moiety with a hydrophobic side chain:
R^{1b} is an optionally substituted amino acid moiety with a polar uncharged side chain;
R^{1c} is an optionally substituted amino acid moiety with a hydrophobic side chain;
R^{2a} is H or optionally substituted alkyl;

### R^{2b} is H or OH; and

R³ is alkyl.

In certain embodiments, R^{1a}, R^{1b}, R^{1c}, R^{2a}, R^{2b}, and R³ may be any of the embodiments or variations described above for formula (I).

In certain variations, the compound of formula (II) is a compound of formula (II-A):

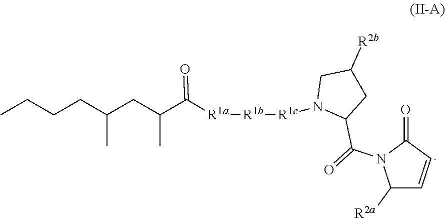

In certain variations, the compound of formula (II) is a compound of formula (II-B):

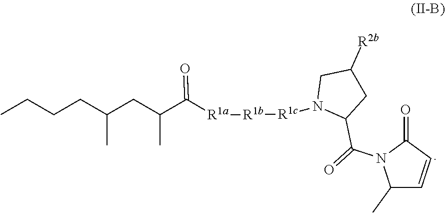

### Compounds of Formula (III)

In other variations, the composition administered comprises: at least one compound of formula (III):

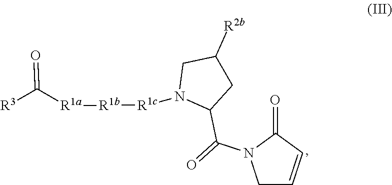

a salt thereof, including any isomers of the foregoing, wherein:
R^{1a} is an optionally substituted amino acid moiety with a hydrophobic side chain;
R^{1b} is an optionally substituted amino acid moiety with a polar uncharged side chain; and
R^{1c} is an optionally substituted amino acid moiety with a hydrophobic side chain.

### R^{2b} is H or OH; and

R³ is alkyl.

In certain embodiments, R^{1a}, R^{1b}, R^{1c}, R^{2b}, and R³ may be any of the embodiments or variations described above for formula (I).

In certain variations, the compound of formula (III) is a compound of formula (III-A):

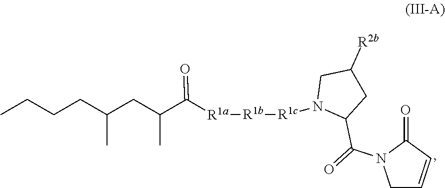

a salt thereof, including any isomers of the foregoing, wherein R^{1a}, R^{1b}, R^{1c}, and R^{2b} are as defined above for formula (III).

### Compounds of Formula (IV)

In other variations, the composition administered comprises: at least one compound of formula (IV):

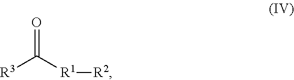

or a salt thereof, including any isomers of the foregoing, wherein:

R¹ is at least one optionally substituted amino acid moiety;

R² is

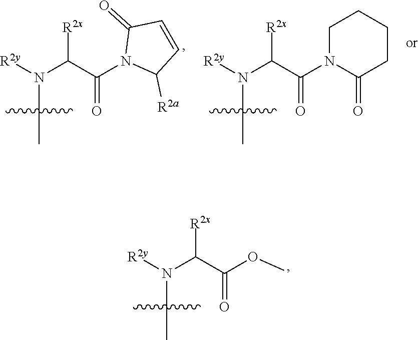

wherein

R^{2a} is H, oxo or optionally substituted alkyl;

R^{2x} is alkyl, optionally substituted with —OH or —COOH;

R^{2y} is H or alkyl; or

R^{2x} and R^{2y} are taken together with the atoms to which they are attached to form an optionally substituted 5-membered heterocycle; and

R³ is alkenyl.

In certain embodiments, R¹, R^{2a}, R^{2x}, R^{2y} and R³ may be any of the embodiments or variations described above for formula (I).

In certain variations, the compound of formula (IV) is a compound of formula (IV-A):

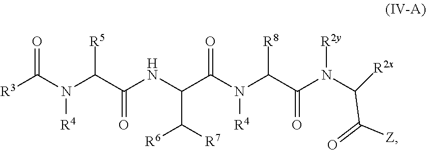

or a salt thereof, including any isomers of the foregoing,
wherein R^{2x}, R^{2y}, and R³ are as defined above for formula (IV), and
wherein:

Z is

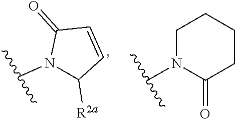

or alkoxy, wherein

R^{2a} is H, oxo or optionally substituted alkyl;

R⁴ is H or alkyl;

each R⁵ and R⁸ is independently a hydrophobic side chain of Ala, Val, Ile, Leu, Met, Phe, Tyr, Trp, or Gly, wherein the hydrophobic side chain is optionally substituted;

R⁶ is H or alkyl; and

R⁷ is —OH, —O(C═O)alkyl, —SH, or —S(C═O)alkyl.

In certain variations, Z is

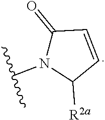

In some embodiments, R^{2a} is H. In certain embodiments, R^{2a} is optionally substituted alkyl. In some variations, R^{2a} is alkyl. In certain variations, R^{2a} is C₁₋₁₀ alkyl, or C₁₋₅ alkyl, or C₁₋₃ alkyl. In one variation, R^{2a} is methyl. In yet other embodiments, R^{2a} is oxo.

In some variations R^{2x} and R^{2y} are taken together with the atoms to which they are attached to form an unsubstituted or substituted 5-membered heterocycle. In certain variations, the heterocycle is pyrrolidine. In some variations R^{2x} and R^{2y} are taken together with the atoms to which they are attached to form 5-membered heterocycle substituted with —OH. In certain variations, the heterocycle is pyrrolidine substituted with —OH. In other variations, the heterocycle is unsubstituted pyrrolidine.

In some variations, R³ is alkenyl. In certain variations, R³ is C₂-C₃₀ alkenyl. In certain variations R³ is

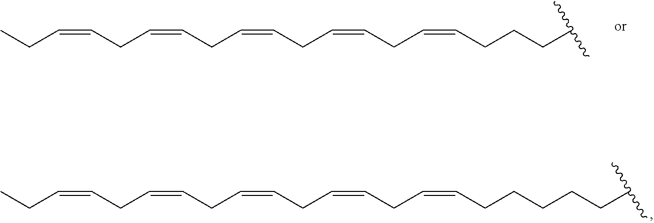 [SMILES: *CCC/C=C\C/C=C\C/C=C\C/C=C\C/C=C\CC ; *CCCCC/C=C\C/C=C\C/C=C\C/C=C\C/C=C\CC]

including any cis- and/or trans-configurations of such moieties.

In yet other variations, R³ is an omega-3 fatty acid, or a derivative thereof. In certain variations, R³ is an eicosapentaenoic acid (EPA) moiety, or a docosahexaenoic acid (DHA) moiety, or a derivative of any of the foregoing.

In certain variations, R⁵ is the hydrophobic side chain of Leu. In certain variations, R⁵ is the hydrophobic side chain of Phe.

In some variations, R⁶ is H. In other variations, R⁶ is alkyl. In certain variations is R⁶ is C₁₋₁₀ alkyl, or C₁₋₅ alkyl, or C₁₋₃ alkyl. In one variation, R^{2a} is methyl.

In certain variations, R⁷ is —OH. In other variations, R⁷ is —SH. In some variations, R⁷ is —O(C═O)alkyl. In some variations, R⁷ is —S(C═O)alkyl. In one variation of the foregoing, the alkyl is methyl.

In certain variations, R⁶ is methyl, and R⁷ is OH.

In one embodiment:

R³ is an omega-3 fatty acid, or a derivative thereof. In certain variations, R³ is an eicosapentaenoic acid (EPA) moiety, or a docosahexaenoic acid (DHA) moiety, or a derivative of any of the foregoing;

R⁵ is the hydrophobic side chain of Phe;

R⁶ is methyl; and

R⁷ is-OH.

### Exemplary Compounds

In certain embodiments, the composition comprises: at least one of the following compounds:

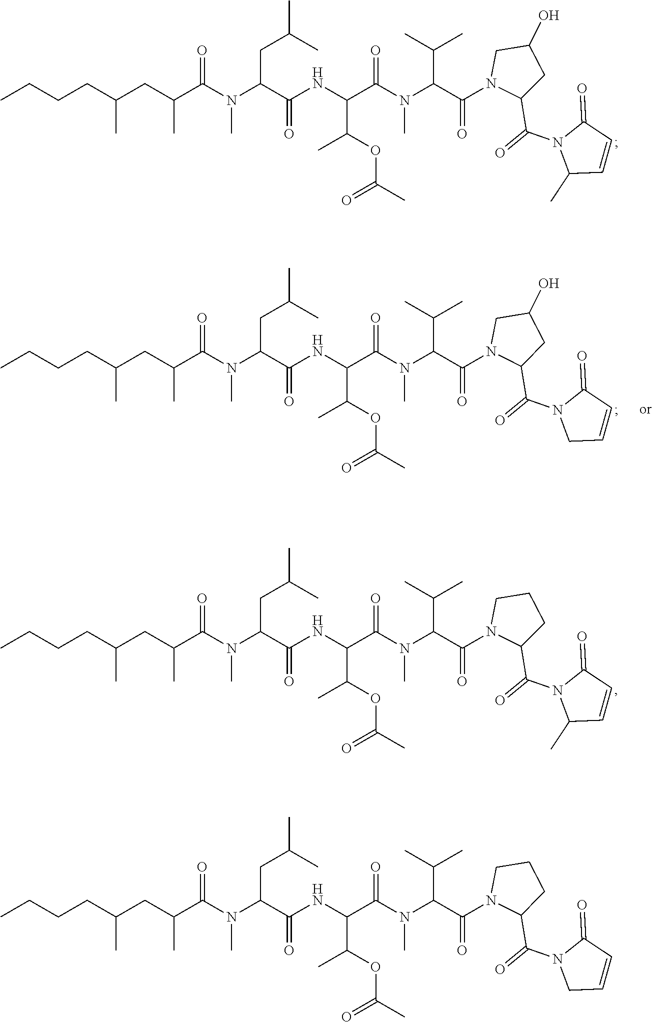 [SMILES: CCCCC(C)CC(C)C(=O)N(C)C(CC(C)C)C(=O)NC(C(=O)N(C)C(C(=O)N1CC(O)CC1C(=O)N1C(=O)C=CC1C)C(C)C)C(C)OC(C)=O ; CCCCC(C)CC(C)C(=O)N(C)C(CC(C)C)C(=O)NC(C(=O)N(C)C(C(=O)N1CC(O)CC1C(=O)N1CC=CC1=O)C(C)C)C(C)OC(C)=O ; CCCCC(C)CC(C)C(=O)N(C)C(CC(C)C)C(=O)NC(C(=O)N(C)C(C(=O)N1CCCC1C(=O)N1C(=O)C=CC1C)C(C)C)C(C)OC(C)=O ; CCCCC(C)CC(C)C(=O)N(C)C(CC(C)C)C(=O)NC(C(=O)N(C)C(C(=O)N1CCCC1C(=O)N1CC=CC1=O)C(C)C)C(C)OC(C)=O]

or a salt thereof, including any isomers of the foregoing.

In one embodiment, the composition comprises: at least one of the following compounds:

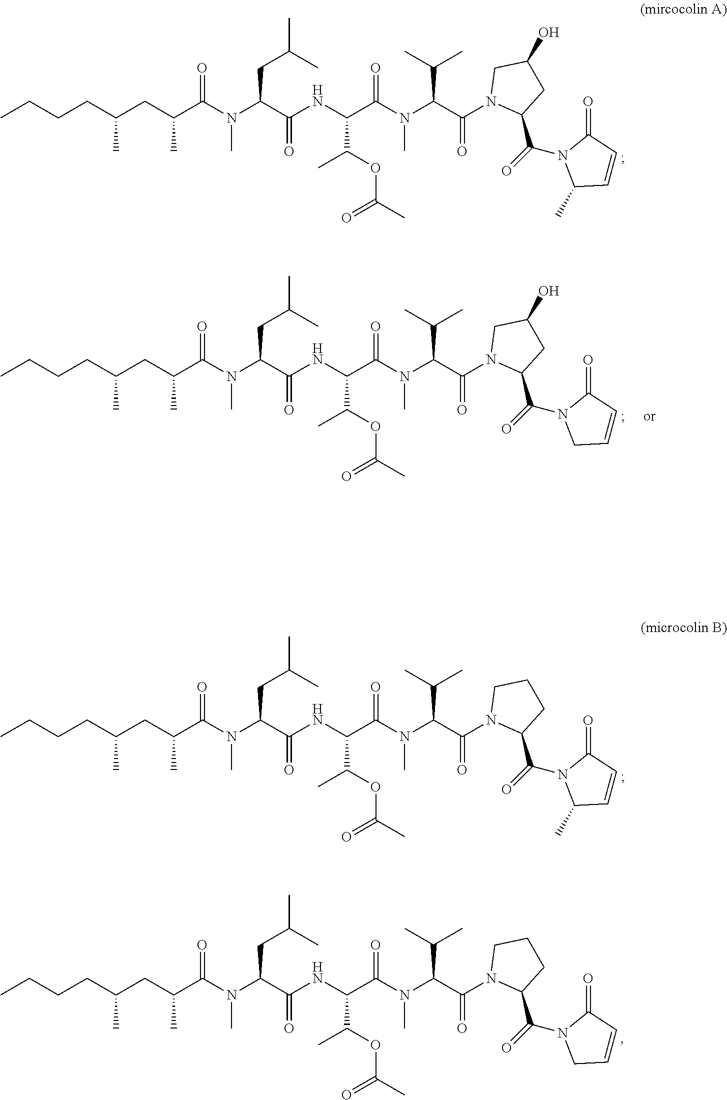

or a salt thereof.

In one embodiment, the composition comprises: microcolin A, or a salt thereof. In some variations, the composition comprises [(2S,3S)-3-[[(2S)-2-[[(2R,4R)-2,4-dimethyloctanoyl]-methylamino]-4-methylpentanoyl]amino]-4-[[(2S)-1-[(2S,4S)-4-hydroxy-2-[(2S)-2-methyl-5-oxo-2H-pyrrole-1-carbonyl]pyrrolidin-1-yl]-3-methyl-1-oxobutan-2-yl]-methylamino]-4-oxobutan-2-yl] acetate, or a salt thereof.

In another embodiment, the composition comprises: microcolin B, or a salt thereof. In some variations, the composition comprises [(2S,3S)-3-[[(2S)-2-[[(2R,4R)-2,4-dimethyloctanoyl]-methylamino]-4-methylpentanoyl]amino]-4-[methyl-[(2S)-3-methyl-1-[(2S)-2-[(2S)-2-methyl-5-oxo-2H-pyrrole-1-carbonyl]pyrrolidin-1-yl]-1-oxobutan-2-yl]amino]-4-oxobutan-2-yl] acetate, or a salt thereof.

In certain embodiments, the composition comprises:

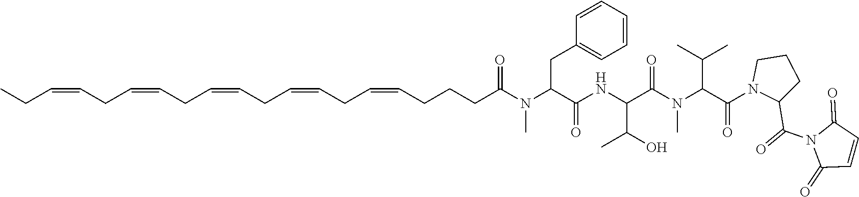 [SMILES: CC/C=C\C/C=C\C/C=C\C/C=C\C/C=C\CCCC(=O)N(C)C(Cc1ccccc1)C(=O)NC(C(=O)N(C)C(C(=O)N1CCCC1C(=O)N1C(=O)C=CC1=O)C(C)C)C(C)O]

or a salt thereof, including any isomers of the foregoing.

In one embodiment, the composition comprises:

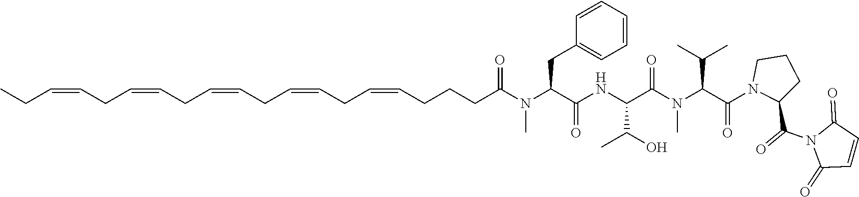 [SMILES: CC/C=C\C/C=C\C/C=C\C/C=C\C/C=C\CCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)O]

or a salt thereof.

In some embodiments, the composition comprises: at least one of the following compounds in Table A below, or any salt thereof.

TABLE A

| Compound | |
|---|---|
| No. | Compound Structure |
| 1 | 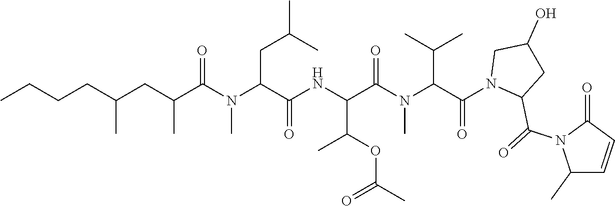 [SMILES: CCCCC(C)CC(C)C(=O)N(C)C(CC(C)C)C(=O)NC(C(=O)N(C)C(C(=O)N1CC(O)CC1C(=O)N1C(=O)C=CC1C)C(C)C)C(C)OC(C)=O] |
| 2 | 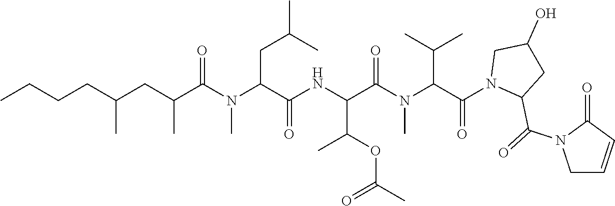 [SMILES: CCCCC(C)CC(C)C(=O)N(C)C(CC(C)C)C(=O)NC(C(=O)N(C)C(C(=O)N1CC(O)CC1C(=O)N1CC=CC1=O)C(C)C)C(C)OC(C)=O] |
| 3 | 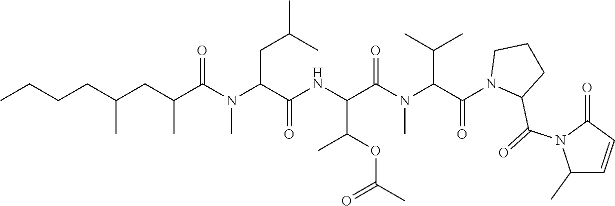 [SMILES: CCCCC(C)CC(C)C(=O)N(C)C(CC(C)C)C(=O)NC(C(=O)N(C)C(C(=O)N1CCCC1C(=O)N1C(=O)C=CC1C)C(C)C)C(C)OC(C)=O] |
| 4 | 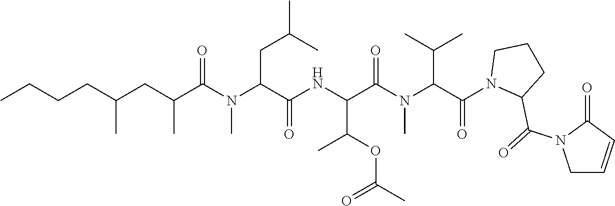 [SMILES: CCCCC(C)CC(C)C(=O)N(C)C(CC(C)C)C(=O)NC(C(=O)N(C)C(C(=O)N1CCCC1C(=O)N1CC=CC1=O)C(C)C)C(C)OC(C)=O] |
| 5 | 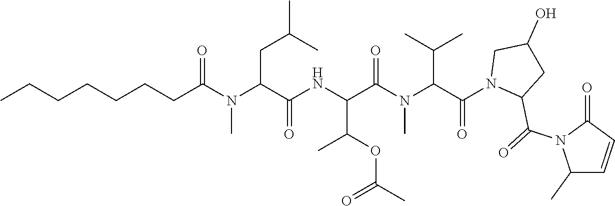 [SMILES: CCCCCCCC(=O)N(C)C(CC(C)C)C(=O)NC(C(=O)N(C)C(C(=O)N1CC(O)CC1C(=O)N1C(=O)C=CC1C)C(C)C)C(C)OC(C)=O] |
| 6 | 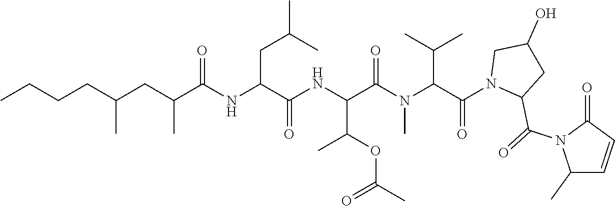 [SMILES: CCCCC(C)CC(C)C(=O)NC(CC(C)C)C(=O)NC(C(=O)N(C)C(C(=O)N1CC(O)CC1C(=O)N1C(=O)C=CC1C)C(C)C)C(C)OC(C)=O] |
| 7 | 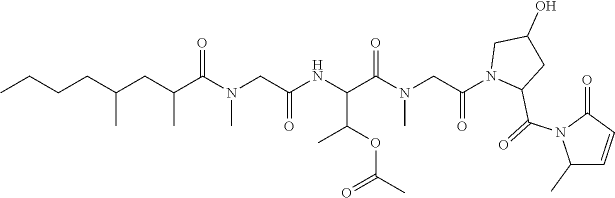 [SMILES: CCCCC(C)CC(C)C(=O)N(C)CC(=O)NC(C(=O)N(C)CC(=O)N1CC(O)CC1C(=O)N1C(=O)C=CC1C)C(C)OC(C)=O] |
| 8 | 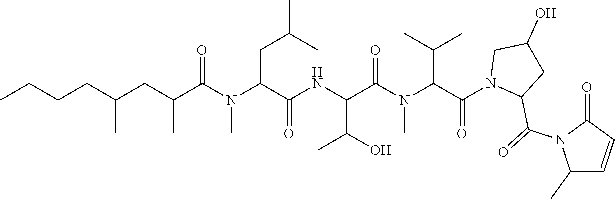 [SMILES: CCCCC(C)CC(C)C(=O)N(C)C(CC(C)C)C(=O)NC(C(=O)N(C)C(C(=O)N1CC(O)CC1C(=O)N1C(=O)C=CC1C)C(C)C)C(C)O] |
| 9 | 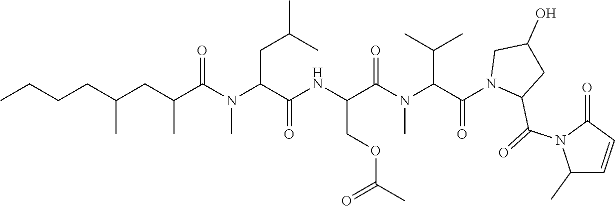 [SMILES: CCCCC(C)CC(C)C(=O)N(C)C(CC(C)C)C(=O)NC(COC(C)=O)C(=O)N(C)C(C(=O)N1CC(O)CC1C(=O)N1C(=O)C=CC1C)C(C)C] |
| 10 | 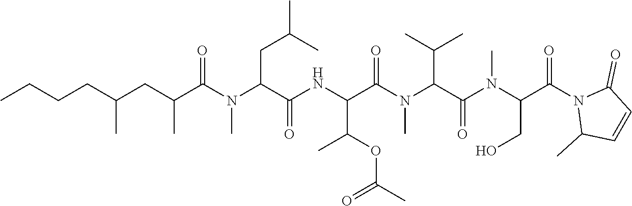 [SMILES: CCCCC(C)CC(C)C(=O)N(C)C(CC(C)C)C(=O)NC(C(=O)N(C)C(C(=O)N(C)C(CO)C(=O)N1C(=O)C=CC1C)C(C)C)C(C)OC(C)=O] |
| 11 | 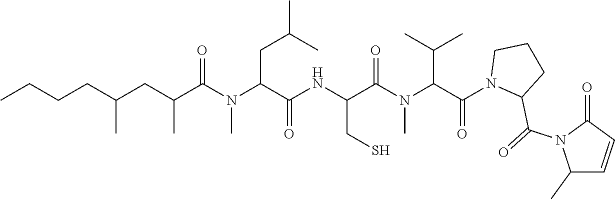 [SMILES: CCCCC(C)CC(C)C(=O)N(C)C(CC(C)C)C(=O)NC(CS)C(=O)N(C)C(C(=O)N1CCCC1C(=O)N1C(=O)C=CC1C)C(C)C] |
| 12 | 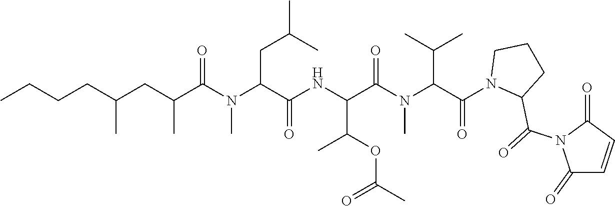 [SMILES: CCCCC(C)CC(C)C(=O)N(C)C(CC(C)C)C(=O)NC(C(=O)N(C)C(C(=O)N1CCCC1C(=O)N1C(=O)C=CC1=O)C(C)C)C(C)OC(C)=O] |
| 13 | 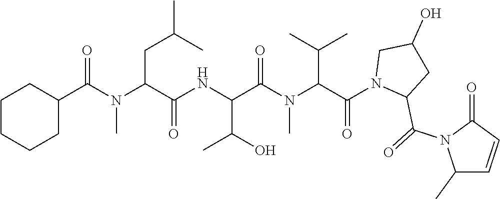 [SMILES: CC(C)CC(C(=O)NC(C(=O)N(C)C(C(=O)N1CC(O)CC1C(=O)N1C(=O)C=CC1C)C(C)C)C(C)O)N(C)C(=O)C1CCCCC1] |
| 14 | 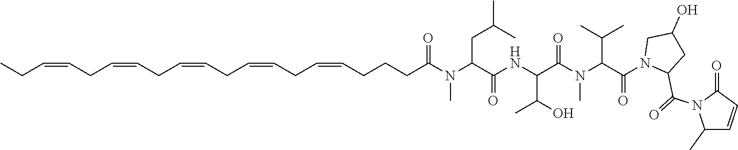 [SMILES: CC/C=C\C/C=C\C/C=C\C/C=C\C/C=C\CCCC(=O)N(C)C(CC(C)C)C(=O)NC(C(=O)N(C)C(C(=O)N1CC(O)CC1C(=O)N1C(=O)C=CC1C)C(C)C)C(C)O] |
| 15 | 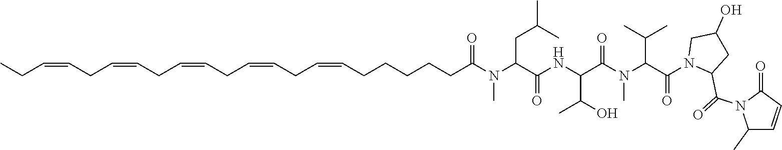 [SMILES: CC/C=C\C/C=C\C/C=C\C/C=C\C/C=C\CCCCCC(=O)N(C)C(CC(C)C)C(=O)NC(C(=O)N(C)C(C(=O)N1CC(O)CC1C(=O)N1C(=O)C=CC1C)C(C)C)C(C)O] |
| 16 | 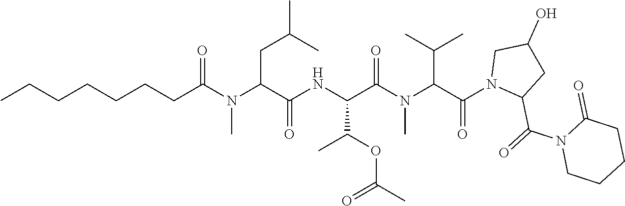 [SMILES: CCCCCCCC(=O)N(C)C(CC(C)C)C(=O)N[C@H](C(=O)N(C)C(C(=O)N1CC(O)CC1C(=O)N1CCCCC1=O)C(C)C)C(C)OC(C)=O] |
| 17 | 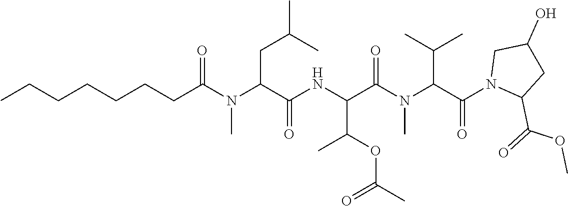 [SMILES: CCCCCCCC(=O)N(C)C(CC(C)C)C(=O)NC(C(=O)N(C)C(C(=O)N1CC(O)CC1C(=O)OC)C(C)C)C(C)OC(C)=O] |
| 18 | 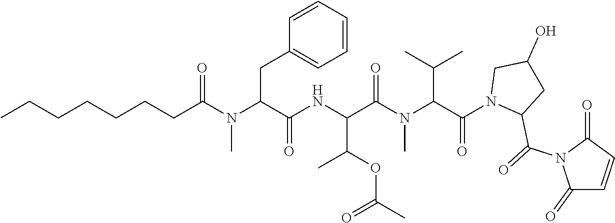 [SMILES: CCCCCCCC(=O)N(C)C(Cc1ccccc1)C(=O)NC(C(=O)N(C)C(C(=O)N1CC(O)CC1C(=O)N1C(=O)C=CC1=O)C(C)C)C(C)OC(C)=O] |
| 19 | 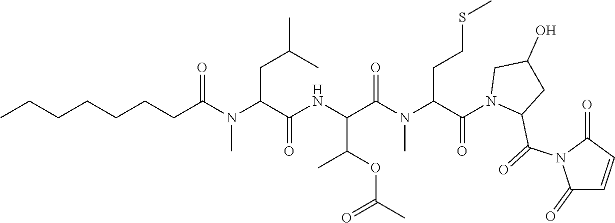 [SMILES: CCCCCCCC(=O)N(C)C(CC(C)C)C(=O)NC(C(=O)N(C)C(CCSC)C(=O)N1CC(O)CC1C(=O)N1C(=O)C=CC1=O)C(C)OC(C)=O] |
| 20 | 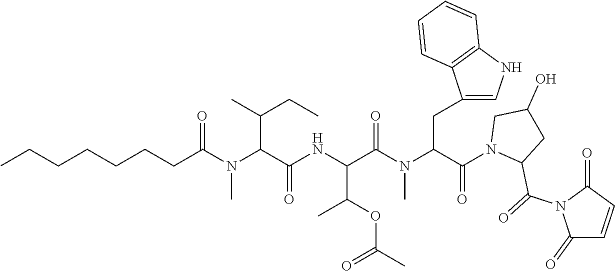 [SMILES: CCCCCCCC(=O)N(C)C(C(=O)NC(C(=O)N(C)C(Cc1c[nH]c2ccccc12)C(=O)N1CC(O)CC1C(=O)N1C(=O)C=CC1=O)C(C)OC(C)=O)C(C)CC] |
| 21 | 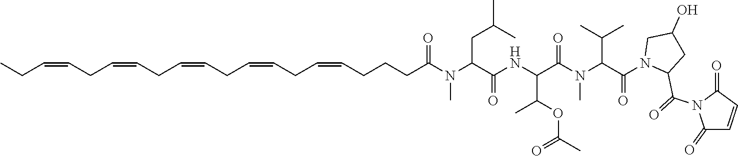 [SMILES: CC/C=C\C/C=C\C/C=C\C/C=C\C/C=C\CCCC(=O)N(C)C(CC(C)C)C(=O)NC(C(=O)N(C)C(C(=O)N1CC(O)CC1C(=O)N1C(=O)C=CC1=O)C(C)C)C(C)OC(C)=O] |
| 22 | 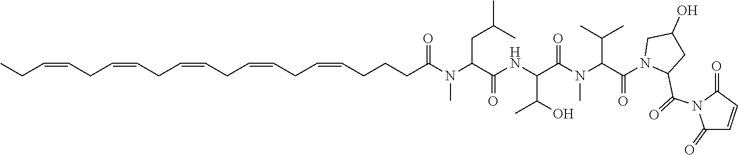 [SMILES: CC/C=C\C/C=C\C/C=C\C/C=C\C/C=C\CCCC(=O)N(C)C(CC(C)C)C(=O)NC(C(=O)N(C)C(C(=O)N1CC(O)CC1C(=O)N1C(=O)C=CC1=O)C(C)C)C(C)O] |
| 23 | 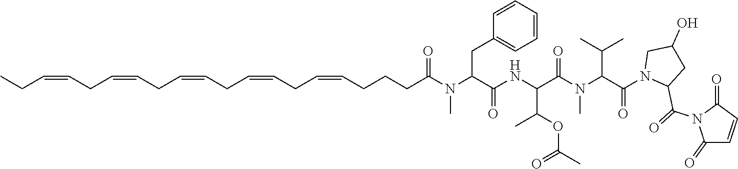 [SMILES: CC/C=C\C/C=C\C/C=C\C/C=C\C/C=C\CCCC(=O)N(C)C(Cc1ccccc1)C(=O)NC(C(=O)N(C)C(C(=O)N1CC(O)CC1C(=O)N1C(=O)C=CC1=O)C(C)C)C(C)OC(C)=O] |
| 24 | 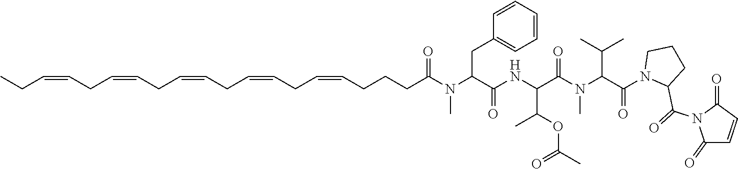 [SMILES: CC/C=C\C/C=C\C/C=C\C/C=C\C/C=C\CCCC(=O)N(C)C(Cc1ccccc1)C(=O)NC(C(=O)N(C)C(C(=O)N1CCCC1C(=O)N1C(=O)C=CC1=O)C(C)C)C(C)OC(C)=O] |
| 25 | 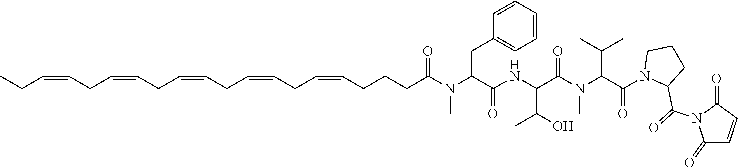 [SMILES: CC/C=C\C/C=C\C/C=C\C/C=C\C/C=C\CCCC(=O)N(C)C(Cc1ccccc1)C(=O)NC(C(=O)N(C)C(C(=O)N1CCCC1C(=O)N1C(=O)C=CC1=O)C(C)C)C(C)O] |
| 26 | 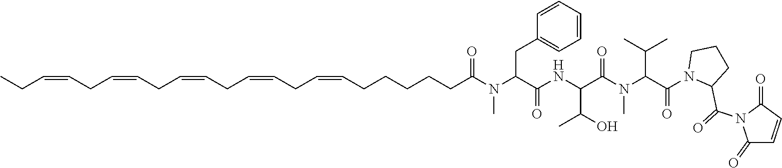 [SMILES: CC/C=C\C/C=C\C/C=C\C/C=C\C/C=C\CCCCCC(=O)N(C)C(Cc1ccccc1)C(=O)NC(C(=O)N(C)C(C(=O)N1CCCC1C(=O)N1C(=O)C=CC1=O)C(C)C)C(C)O] |
| 27 | 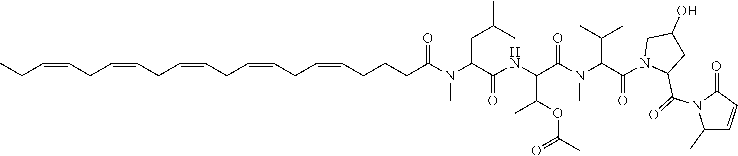 [SMILES: CC/C=C\C/C=C\C/C=C\C/C=C\C/C=C\CCCC(=O)N(C)C(CC(C)C)C(=O)NC(C(=O)N(C)C(C(=O)N1CC(O)CC1C(=O)N1C(=O)C=CC1C)C(C)C)C(C)OC(C)=O] |

In some embodiments, the composition comprises: at least one of the following compounds in Table A′ below, or any salt thereof.

TABLE A′

| Compound | |
|---|---|
| No. | Compound Structure |
| 1a | 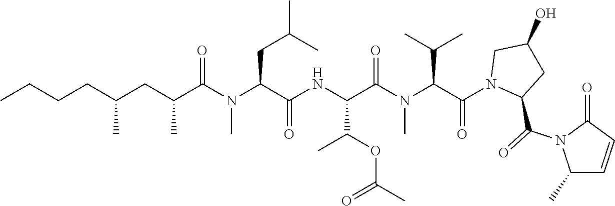 [SMILES: CCCC[C@@H](C)C[C@@H](C)C(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O] |
| 1b | 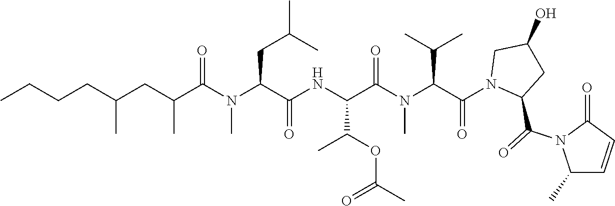 [SMILES: CCCCC(C)CC(C)C(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O] |
| 2a | 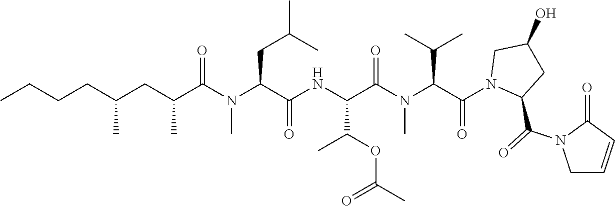 [SMILES: CCCC[C@@H](C)C[C@@H](C)C(=O)N(C)[C@@H](CC(C)C)C(=O)N[C@H](C(=O)N(C)[C@H](C(=O)N1C[C@@H](O)C[C@H]1C(=O)N1CC=CC1=O)C(C)C)C(C)OC(C)=O] |
| 3a | 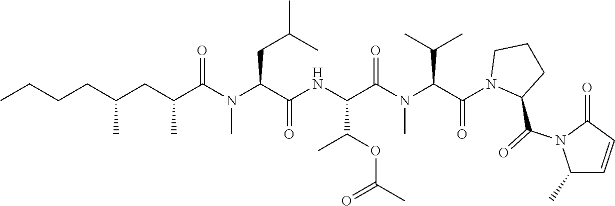 [SMILES: CCCC[C@@H](C)C[C@@H](C)C(=O)N(C)[C@@H](CC(C)C)C(=O)N[C@H](C(=O)N(C)[C@H](C(=O)N1CCC[C@H]1C(=O)N1C(=O)C=C[C@@H]1C)C(C)C)C(C)OC(C)=O] |
| 4a | 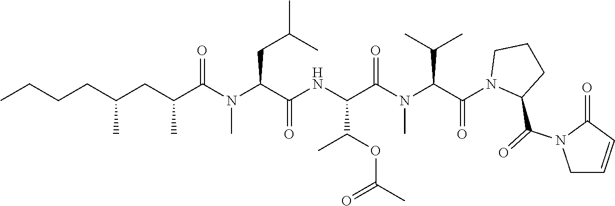 [SMILES: CCCC[C@@H](C)C[C@@H](C)C(=O)N(C)[C@@H](CC(C)C)C(=O)N[C@H](C(=O)N(C)[C@H](C(=O)N1CCC[C@H]1C(=O)N1CC=CC1=O)C(C)C)C(C)OC(C)=O] |
| 5a | 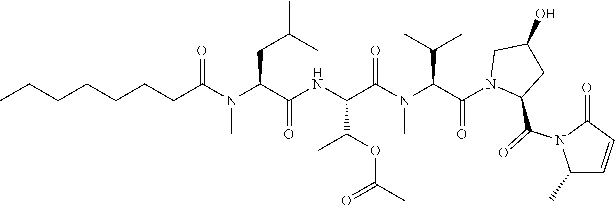 [SMILES: CCCCCCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O] |
| 6a | 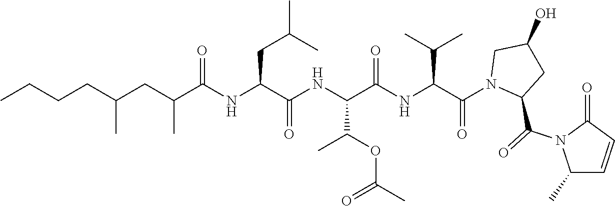 [SMILES: CCCCC(C)CC(C)C(=O)N[C@@H](CC(C)C)C(=O)N[C@H](C(=O)N[C@H](C(=O)N1C[C@@H](O)C[C@H]1C(=O)N1C(=O)C=C[C@@H]1C)C(C)C)C(C)OC(C)=O] |
| 7a | 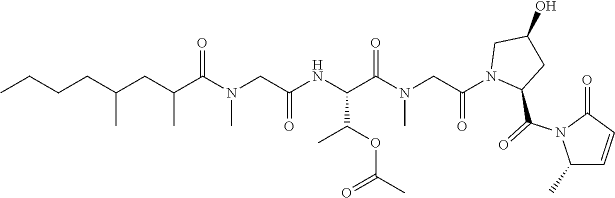 [SMILES: CCCCC(C)CC(C)C(=O)N(C)CC(=O)N[C@H](C(=O)N(C)CC(=O)N1C[C@@H](O)C[C@H]1C(=O)N1C(=O)C=C[C@@H]1C)C(C)OC(C)=O] |
| 8a | 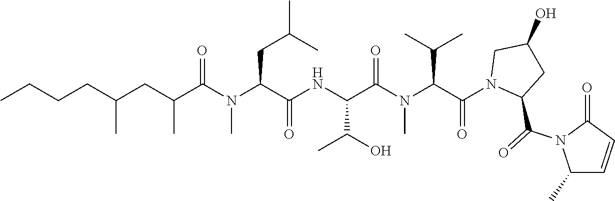 [SMILES: CCCCC(C)CC(C)C(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O] |
| 9a | 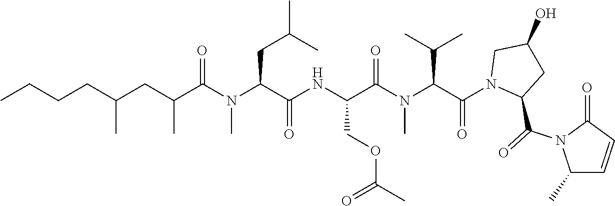 [SMILES: CCCCC(C)CC(C)C(=O)N(C)[C@@H](CC(C)C)C(=O)N[C@@H](COC(C)=O)C(=O)N(C)[C@H](C(=O)N1C[C@@H](O)C[C@H]1C(=O)N1C(=O)C=C[C@@H]1C)C(C)C] |
| 10a | 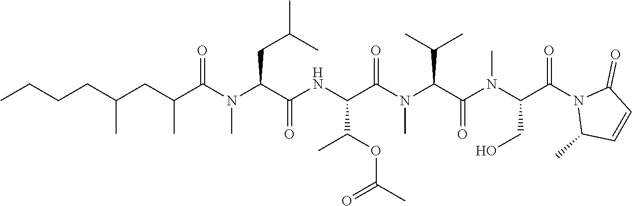 [SMILES: CCCCC(C)CC(C)C(=O)N(C)[C@@H](CC(C)C)C(=O)N[C@H](C(=O)N(C)[C@H](C(=O)N(C)[C@@H](CO)C(=O)N1C(=O)C=C[C@@H]1C)C(C)C)C(C)OC(C)=O] |
| 11a | 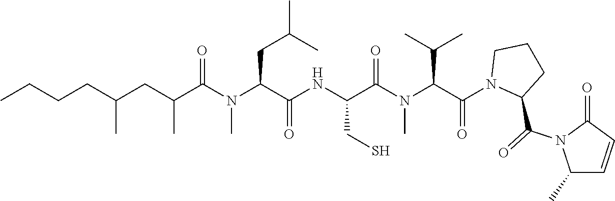 [SMILES: CCCCC(C)CC(C)C(=O)N(C)[C@@H](CC(C)C)C(=O)N[C@@H](CS)C(=O)N(C)[C@H](C(=O)N1CCC[C@H]1C(=O)N1C(=O)C=C[C@@H]1C)C(C)C] |
| 12a | 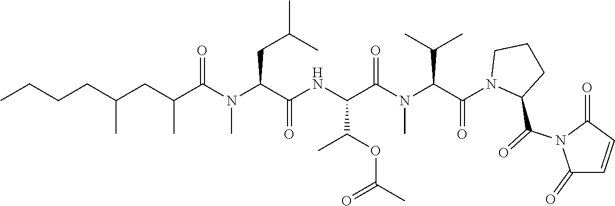 [SMILES: CCCCC(C)CC(C)C(=O)N(C)[C@@H](CC(C)C)C(=O)N[C@H](C(=O)N(C)[C@H](C(=O)N1CCC[C@H]1C(=O)N1C(=O)C=CC1=O)C(C)C)C(C)OC(C)=O] |
| 13a | 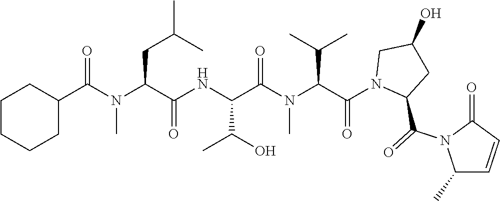 [SMILES: CC(C)C[C@@H](C(=O)N[C@H](C(=O)N(C)[C@H](C(=O)N1C[C@@H](O)C[C@H]1C(=O)N1C(=O)C=C[C@@H]1C)C(C)C)C(C)O)N(C)C(=O)C1CCCCC1] |
| 14a | 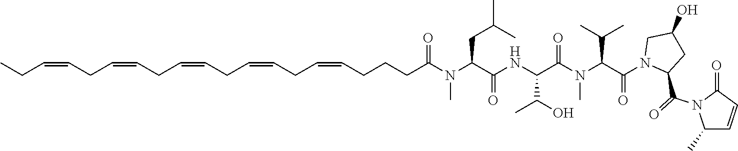 [SMILES: CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O] |
| 15a | 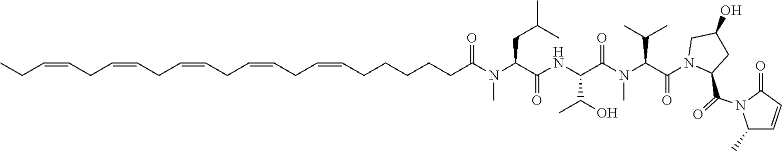 [SMILES: CC/C=C\C/C=C\C/C=C\C/C=C\C/C=C\CCCCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O] |
| 16a | 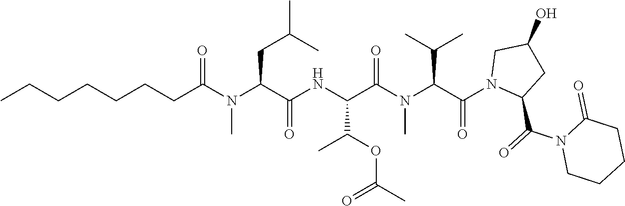 [SMILES: CCCCCCCC(=O)N(C)[C@@H](CC(C)C)C(=O)N[C@H](C(=O)N(C)[C@H](C(=O)N1C[C@@H](O)C[C@H]1C(=O)N1CCCCC1=O)C(C)C)C(C)OC(C)=O] |
| 17a | 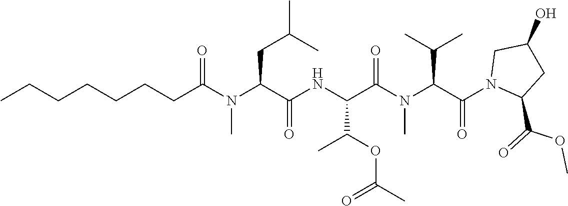 [SMILES: CCCCCCCC(=O)N(C)[C@@H](CC(C)C)C(=O)N[C@H](C(=O)N(C)[C@H](C(=O)N1C[C@@H](O)C[C@H]1C(=O)OC)C(C)C)C(C)OC(C)=O] |
| 18a | 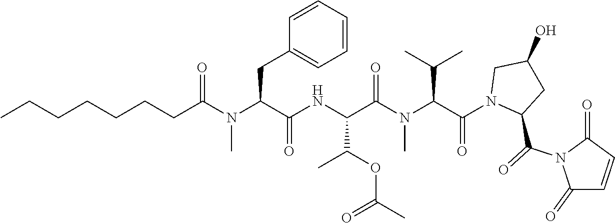 [SMILES: CCCCCCCC(=O)N(C)[C@@H](Cc1ccccc1)C(=O)N[C@H](C(=O)N(C)[C@H](C(=O)N1C[C@@H](O)C[C@H]1C(=O)N1C(=O)C=CC1=O)C(C)C)C(C)OC(C)=O] |
| 19a | 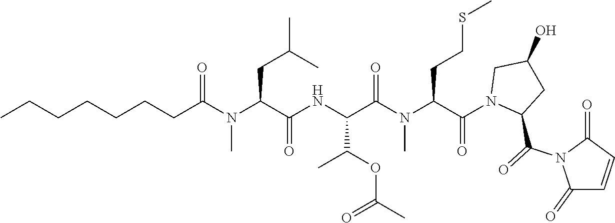 [SMILES: CCCCCCCC(=O)N(C)[C@@H](CC(C)C)C(=O)N[C@H](C(=O)N(C)[C@@H](CCSC)C(=O)N1C[C@@H](O)C[C@H]1C(=O)N1C(=O)C=CC1=O)C(C)OC(C)=O] |
| 20a | 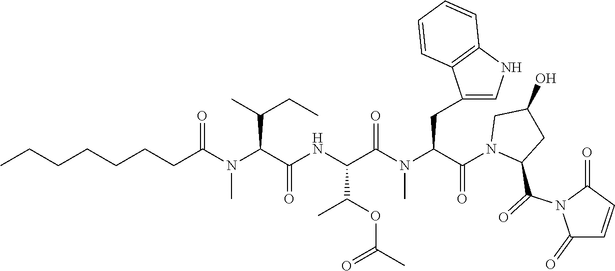 [SMILES: CCCCCCCC(=O)N(C)[C@H](C(=O)N[C@H](C(=O)N(C)[C@@H](Cc1c[nH]c2ccccc12)C(=O)N1C[C@@H](O)C[C@H]1C(=O)N1C(=O)C=CC1=O)C(C)OC(C)=O)C(C)CC] |
| 21a | 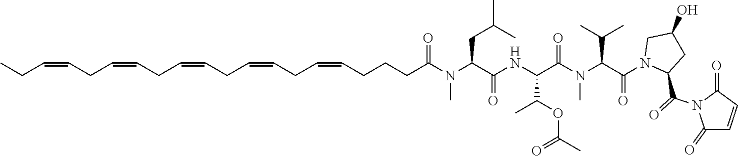 [SMILES: CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=CC1=O)C(C)C)C(C)OC(C)=O] |
| 22a | 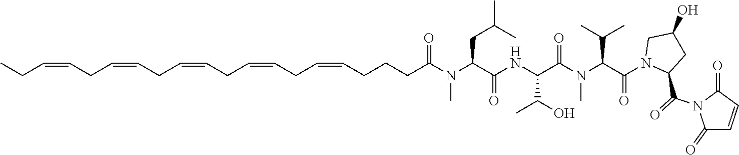 [SMILES: CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=CC1=O)C(C)C)C(C)O] |
| 23a | 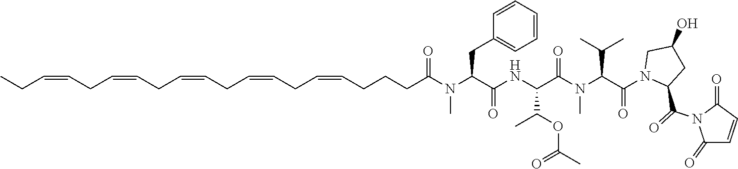 [SMILES: CC/C=C\C/C=C\C/C=C\C/C=C\C/C=C\CCCC(=O)N(C)[C@@H](Cc1ccccc1)C(=O)N[C@H](C(=O)N(C)[C@H](C(=O)N1C[C@@H](O)C[C@H]1C(=O)N1C(=O)C=CC1=O)C(C)C)C(C)OC(C)=O] |
| 24a | 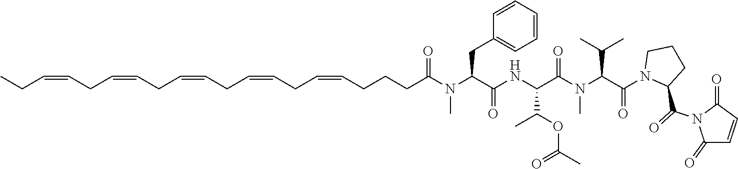 [SMILES: CC/C=C\C/C=C\C/C=C\C/C=C\C/C=C\CCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)OC(C)=O] |
| 25a | 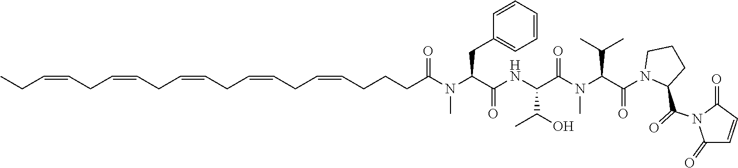 [SMILES: CC/C=C\C/C=C\C/C=C\C/C=C\C/C=C\CCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)O] |
| 26a | 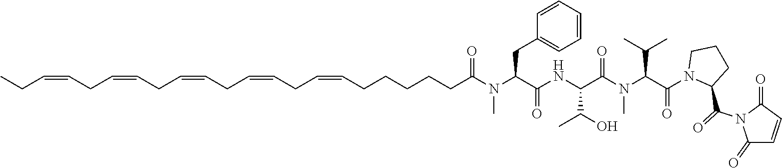 [SMILES: CC/C=C\C/C=C\C/C=C\C/C=C\C/C=C\CCCCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)O] |
| 27a | 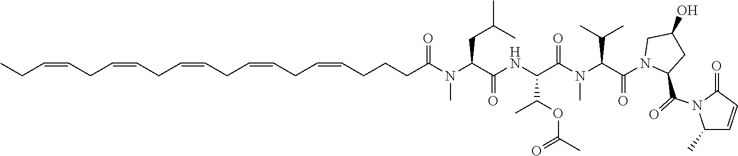 [SMILES: CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O] |

In some variations, the compositions described herein may include one or more isomers of the compounds described herein, including compounds of formulae (I), (II), (II-A), (II-B), (III), (III-A), (IV) and (IV-A), as well as compounds of Tables A and A′. Compounds that have the same molecular formula but differ in the nature or sequence of bonding of their atoms or the arrangement of their atoms in space are termed “isomers.” Isomers that have the same sequence of bonding of their atoms but differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory. The prefixes d and l or (+) and (−) are employed to designate the sign of rotation of plane-polarized light by the compound, with (−) or l meaning that the compound is levorotatory. A compound prefixed with (+) or d is dextrorotatory. A chiral compound can exist as either individual enantiomer or as a mixture thereof. A mixture containing equal proportions of the enantiomers is referred to as a “racemic mixture” or “racemate”.

In certain embodiments, the compositions described herein comprise a racemic mixture of a compound of formula (I), (II), (II-A), (II-B), (III), (III-A), (IV) or (IV-A), or compounds of Tables A and A′. In certain embodiments, the compound is enriched by at least about 90% by weight with a single diastereomer or enantiomer. In other embodiments, the compound is enriched by at least about 95%, 98%, or 99% by weight with a single diastereomer or enantiomer.

In some variations, the compositions described herein may include one or more salts of the compounds described herein, including compounds of formulae (I), (II), (II-A), (II-B), (III), (III-A), (IV) and (IV-A), as well as compounds of Tables A and A′. In certain variations, the salts are pharmaceutically acceptable salts. The term “pharmaceutically acceptable salts” refers to those salts which retain the biological effectiveness and properties of the free bases or free acids, which are not biologically or otherwise undesirable.

### General Synthesis for Compounds

The compounds provided herein, including compounds of formulae (I), (II), (II-A), (II-B), (III), (III-A), (IV) and (IV-A) and the exemplary compounds of Tables A and A′, may be synthesized according to General Scheme 1 below. The general scheme, as well as the reactions described in the Examples section below, can be readily adapted to prepare the compounds disclosed herein. For example, the synthesis of non-exemplified compounds according to the present disclosure can be successfully performed by modifications apparent to those skilled in the art, e.g., by utilizing other suitable reagents known in the art other than those described, or by making routine modifications of reaction conditions, reagents, and starting materials.

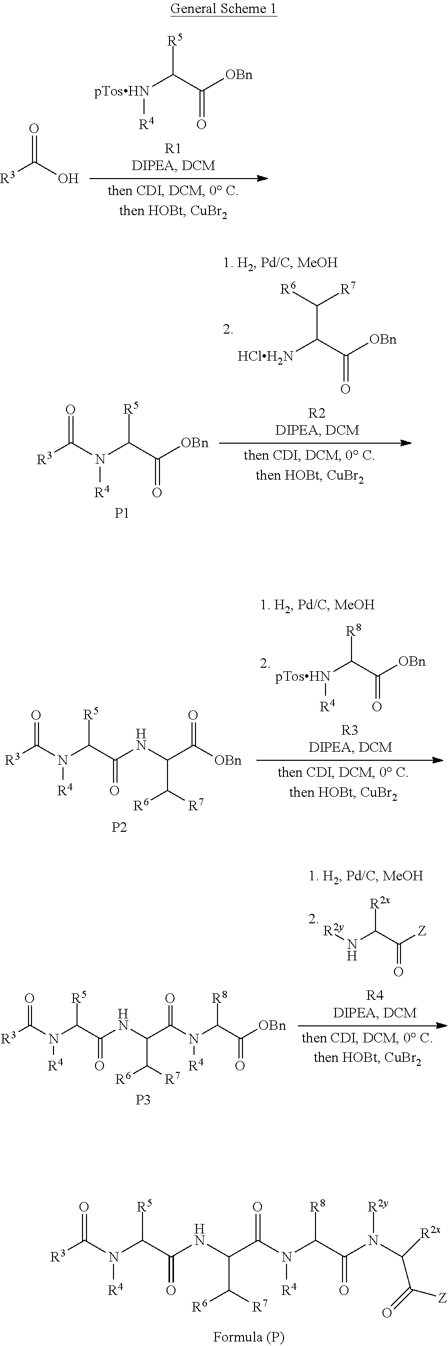

The exemplary compounds of Tables A and A′, may be synthesized according to General Scheme 1 above, wherein:

Z is

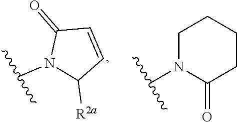

or alkoxy, wherein

R^{2a} is H, oxo or optionally substituted alkyl;

R^{2x} is alkyl, optionally substituted with —OH or —COOH;

R^{2y} is H or alkyl; or

R^{2x} and R^{2y} are taken together with the atoms to which they are attached to form an optionally substituted 5-membered heterocycle;

R³ is alkyl, cycloalkyl, or alkenyl;

R⁴ is H or alkyl;

each R⁵ and R⁸ is independently a hydrophobic side chain of Ala, Val, Ile, Leu, Met, Phe, Tyr, Trp, or Gly, wherein the hydrophobic side chain is optionally substituted;

R⁶ is H or alkyl; and

R⁷ is —OH, —O(C═O)alkyl, —SH, or —S(C═O)alkyl.

The general scheme, as well as the reactions described in the Examples section below, can be readily adapted to prepare the compounds disclosed herein. For example, in some variations, reagent R1 may be N-Methyl-L-leucine benzyl ester p-toluenesulfonate or may be substituted with other appropriate compounds to produce other moieties corresponding to variables R⁴ and R⁵ in formula (IV-A).

An exemplary procedure for the general scheme (Scheme 1) is provided below:

Step 1 (Condensation of R³COOH). A coupling reagent (e.g. N,N′-carbonyldiimidazole (CDI)) is combined with a suitable solvent, such as dichloromethane. This solution is cooled. Separately, R¹ is also combined with a suitable solvent, such as dichloromethane, to which a suitable base, such as diisopropylethylamine (DIPEA), is added. Once the suspension turns clear, it is added to the chilled CDI suspension. Once the resulting second suspension turns clear, the reaction continues under drying conditions (such as under a CaCl₂ drying tube) and is continued until completion. Upon completion, the reaction mixture undergoes a workup, involving extraction with a suitable organic solvent, such as ethyl acetate, and the organic layer dried (e.g., over MgSO₄), filtered and solvent is removed to obtain a crude product (such as a viscous oil).

To this crude product, additional solvent, such as dichloromethane, is added. To this, R³COOH, a coupling reagent, such as 1-hydroxybenzotriazole hydrate (HOBt hydrate), and a catalyst, such as CuBr₂. Upon completion of the reaction, the reaction mixture undergoes a workup, involving quenching (e.g. with an aqueous solution of HCl) and extraction with a suitable organic solvent, such as dichloromethane, and the organic layer is washed with an aqueous solution (e.g. NaHCO₃), dried (e.g. over MgSO₄), filtered and the solvent is removed to obtain a crude product (such as an oil) (P1).

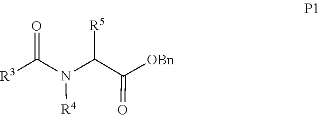

Step 2 (Condensation of P1 with R2). P1 is added to an organic solvent (e.g. methanol) and a catalyst (e.g. Pd/C). The reaction is stirred under H₂, then filtered and the solvent is removed to obtain a crude product (e.g. an oil) (P1 acid).

A coupling reagent (e.g. N,N′-carbonyldiimidazole (CDI)) is combined with a suitable solvent, such as dichloromethane. This solution is cooled. Separately, R2 is also combined with a suitable solvent, such as dichloromethane to which a suitable base, such as diisopropylethylamine (DIPEA), is added. Once the suspension turns clear, it is added to the chilled CDI suspension. Once the resulting second suspension turns clear, the reaction continues under drying conditions (such as under a CaCl₂) drying tube) and is continued until completion. Upon completion, the reaction mixture undergoes a workup, involving extraction with a suitable organic solvent, such as ethyl acetate, and the organic layer dried (e.g., over MgSO₄), filtered and solvent is removed to obtain a crude product (such as a viscous oil).

To this crude product, additional solvent, such as dichloromethane, is added. To this, P1 acid (from above), a coupling reagent, such as 1-hydroxybenzotriazole hydrate (HOBt hydrate), and a catalyst, such as CuBr₂. Upon completion of the reaction, the reaction mixture undergoes a workup, involving quenching (e.g. with an aqueous solution of HCl) and extraction with a suitable organic solvent, such as dichloromethane, and the organic layer is washed with an aqueous solution (e.g. NaHCO₃), dried (e.g. over MgSO₄), filtered and the solvent is removed to obtain a crude product (such as an oil) (P2).

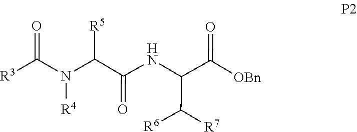

Step 3 (Condensation of P2 with R3). P2 is added to a suitable organic solvent, such as methanol, and a suitable catalyst, such as Pd/C. The reaction is stirred under H₂, then filtered and the solvent is removed to obtain a crude product (e.g. an oil) (P2 acid).

A coupling reagent (e.g. N,N′-carbonyldiimidazole (CDI)) is combined with a suitable solvent, such as dichloromethane. This solution is cooled. Separately, R3 is also combined with a suitable solvent, such as dichloromethane to which a suitable base, such as diisopropylethylamine (DIPEA), is added. Once the suspension turns clear, it is added to the chilled CDI suspension. Once the resulting second suspension turns clear, the reaction continues under drying conditions (such as under a CaCl₂ drying tube) and is continued until completion. Upon completion, the reaction mixture undergoes a workup, involving extraction with a suitable organic solvent, such as ethyl acetate, and the organic layer dried (e.g., over MgSO₄), filtered and solvent is removed to obtain a crude product (such as a viscous oil).

To this crude product, additional solvent, such as dichloromethane, is added. To this, P2 acid (from above), a suitable coupling reagent, such as 1-hydroxybenzotriazole hydrate (HOBt hydrate), and a suitable catalyst, such as CuBr₂. Upon completion of the reaction, the reaction mixture undergoes a workup, involving quenching (e.g. with an aqueous solution of HCl) and extraction with a suitable organic solvent, such as dichloromethane, and the organic layer is washed with an aqueous solution (e.g. NaHCO₃), dried (e.g. over MgSO₄), filtered and the solvent is removed to obtain a crude product (such as an oil) (P3).

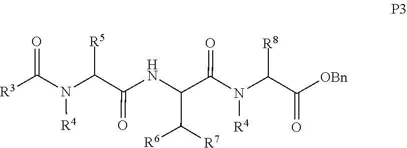

Step 4 (Condensation of P3 with cis-hydroxyproline 4-methylpyrrolidinone amide). P3 is added to a suitable organic solvent, such as methanol, and a suitable catalyst, such as Pd/C. The reaction is stirred under H₂, then filtered and the solvent is removed to obtain a crude product (e.g. an oil) (P3 acid).

A suitable coupling reagent, such as N,N′-carbonyldiimidazole (CDI), is combined with a suitable solvent, such as dichloromethane. This solution is cooled. Separately, R4 is also combined with a suitable solvent, such as dichloromethane to which a suitable base, such as diisopropylethylamine (DIPEA), is added. Once the suspension turns clear, it is added to the chilled CDI suspension. Once the resulting second suspension turns clear, the reaction continues under drying conditions (such as under a CaCl₂) drying tube) and is continued until completion. Upon completion, the reaction mixture undergoes a workup, involving extraction with a suitable organic solvent, such as ethyl acetate, and the organic layer dried (e.g., over MgSO₄), filtered and solvent is removed to obtain a crude product (such as a viscous oil).

To this crude product, additional solvent, such as dichloromethane, is added. To this, P3 acid (from above), a coupling reagent, such as 1-hydroxybenzotriazole hydrate (HOBt hydrate), and a catalyst, such as CuBr₂. Upon completion of the reaction, the reaction mixture undergoes a workup, involving quenching (e.g. with an aqueous solution of HCl) and extraction with a suitable organic solvent, such as dichloromethane, and the organic layer is washed with an aqueous solution (e.g. NaHCO₃), dried (e.g. over MgSO₄), filtered and the solvent is removed to obtain a crude product (such as an oil) (Formula P).

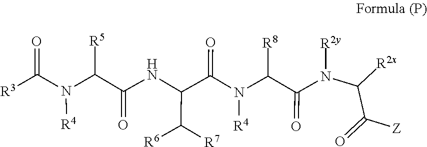

### Other Components

In some embodiments, the compositions for the respiratory treatments further include one or more additional components. In some variations, the additional components include minor fatty acid triglyceride components. In some variations, the additional components include saturated acids. In certain variations, the additional components include caproic acid, caprylic acid, capric acid, lauric acid, behenic acid, or lignoceric acid, or any combination thereof.

In some variations, the additional components include monounsaturated acids. In certain variations, the additional components include myrstoleic acid, heptadecenoic acid, elaidic acid, gadoleic acid, erucic acid, brassidic acid, and/or nervonic acid.

In some variations, the additional components include polyunsaturated acids. In certain variations, the additional components include gamma linolenic acid, columbinic acid, stearidonic acid, mead acid, and/or dihomo gamma linolenic acid.

In other variations, the additional components include small organic molecules. In some variations, the additional components include terpenes (e.g., ligustilide), sesquiterpenes (e.g., germacrene), phenols (e.g., thymol, eugenol, carvacrol), alcohols (e.g., linalool, citronellol, terpineol), sesquiterpene alcohols (e.g., bisbalol, santalol), ketones (e.g., thujone, pinacamphone, italidone), esters (e.g., linalyl acetate, geranyl acetate, citronellyl formate), lactones and coumarins (e.g., helenalin, elecampane, furocoumarin), ethers (e.g., chavicol), steroid derivatives (e.g., sitosterol, stigmasterol), and/or phthalide derivatives (e.g., 3-butyliden-4,5-dihydrophthalide).

In some variations, the additional components include other lipopeptides. In some variations, the additional components include linear and/or cyclic lipopeptides. In certain variations, the additional components include iturin A, hoiamides, heronamides, laxaphycin, apramides, dragonamides, gageotetrins, lyngbyabellins, cyclodycidins, parguerine, pumilacidin, sulforeido lipopeptides, fengycins, mebamamides, penicimutamides, sulfoglycolipids, halovir, kahalalide, and/or tuftsin.

In some variations of the foregoing additional components described, the compound(s) may be present in salt form, including in pharmaceutically acceptable salt forms.

### Conditions, Diseases or Disorders

In some aspects, the compositions provided herein, including compositions comprising compounds of formulae (I), (II), (II-A), (II-B), (I), (III-A), (IV) and (IV-A), may be used to treat inflammatory conditions, disorders or diseases, including respiratory conditions, disorders or diseases. In some embodiments, the conditions, disorders or diseases are inflammations of the respiratory tract. In some aspects, provided is a method for treating inflammatory conditions, disorders or diseases in a human in need thereof, comprising administering the compositions provided herein to the human.

In some variations, “treatment” or “treating” is an approach for obtaining beneficial or desired results including clinical results. Beneficial or desired clinical results may include one or more of the following:
(i) decreasing one more symptoms resulting from the condition, disease or disorder;
(ii) diminishing the extent of the disease and/or stabilizing the condition, disease or disorder (e.g., delaying the worsening of the condition, disease or disorder);
(iii) delaying the spread of the condition, disease or disorder;
(iv) delaying or slowing the recurrence of the condition, disease or disorder and/or the progression of the condition, disease or disorder;
(v) ameliorating the disease state and/or providing a remission (whether partial or total) of the condition, disease or disorder and/or decreasing the dose of one or more other medications required to treat the condition, disease or disorder;
(vi) increasing the quality of life; and/or
(vii) prolonging survival.

In some embodiments, the compositions provided herein, including compositions comprising compounds of formulae (I), (II), (II-A), (II-B), (III), (III-A), (IV) and (IV-A), may be used to treat asthma, pneumonia, bronchiectasis, emphysema, tuberculosis, lung collapse, lung fibrosis, fibrosing alveolitis, chronic obstructive pulmonary disease (COPD), allergic rhinitis, chronic rhinosinusitis (CRS), and acute respiratory disease syndrome.

In some embodiments, the condition, disease or disorder is a chronic inflammatory disorder. In certain embodiments, the chronic inflammatory disorder is a chronic inflammatory disorder of the airways. In certain variations, the condition, disease or disorder is an inflammatory lung disease. In some variations, the condition, disease or disorder involves narrowing and/or swelling of airways, thereby making breathing difficult and triggering coughing, wheezing and/or shortness of breath. In certain variations, the condition, disease or disorder is asthma. In certain variations, the asthma is bronchial asthma. In one variation, the condition, disease or disorder involves steroid treatment resistant asthma and airway constrictions.

In other embodiments, the condition, disease or disorder is an allergy or an allergic inflammation.

In other embodiments, the condition, disease or disorder is a viral respiratory disease. In some variations, condition, disease or disorder is severe acute respiratory syndrome. In certain variations, the severe acute respiratory syndrome is caused by a coronavirus.

In some variations, the human in need thereof is a lung-compromised individual. In certain variations, the lung-compromised individual has fluid build-up in the alveoli in the lungs. This fluid can leak from the smallest blood vessels in the lungs into the alveoli due to the destruction of the protective membrane in the alveoli. The membrane which normally keeps this fluid in the vessels may be destroyed because of a disruption in immune response due to severe disease or injury. The fluid enters the alveoli and keeps the lungs from filling with enough air, which means less oxygen reaches the bloodstream. This deprives organs of the oxygen that is needed to function, which can cause multiple organ failure resulting in death.

In one embodiment, provided is a method for treating hospitalized lung-compromised humans in need thereof, comprising administering the compositions provided herein, including compositions comprising compounds of formulae (I), (II), (II-A), (II-B), (III), (III-A), (IV) and (IV-A), to the human to reduce or delay the need to provide the human with assisted respiration.

In some variations, “delaying” development of a condition, disease or disorder means to defer, hinder, slow, retard, stabilize and/or postpone development of the condition, disease or disorder. This delay can be of varying lengths of time, depending on the history of the condition, disease or disorder and/or individual being treated.

In other aspects, the compositions provided herein, including compositions comprising compounds of formulae (I), (II), (II-A), (II-B), (III), (III-A), (IV) and (IV-A), improve anti-inflammatory efficacy via a reduction in eosinophil effector function. Thus, in certain embodiments, provided is a method for reducing eosinophil effector function in a human in need thereof, comprising administering the compositions provided herein, including compositions comprising compounds of formulae (I), (II), (II-A), (II-B), (III), (III-A), (IV) and (IV-A), to the human to reduce eosinophil effector function.

### Sub-Patient Population

In some embodiments, the methods provided herein involve treating a human in need thereof. In certain embodiments, the human is largely resistant to medical and surgical interventions for treating the inflammatory conditions, disorders or diseases described herein. In one embodiment, the human exhibits or has resistance to steroid therapy. For example, in one variation, the human has steroid treatment resistant asthma.

In some variations of the foregoing, the human is a child. In certain variations, the human is less than 18 years old, less than 12 years old, less than 10 years old, less than 5 years old, less than 2 years old, or less than 1 year; or between 2 years old and 12 years old.

### Formulations

In some embodiments, the compositions provided herein, including compositions comprising compounds of formulae (I), (II), (II-A), (II-B), (III), (III-A), (IV) and (IV-A), are formulated for oral administration. Forms suitable for oral administration may include, for example, tablets, pills, capsules, cachets, dragees, lozenges, liquids, gels, syrups, slurries, elixirs, suspensions, aerosols, or powders.

In certain embodiments, the pharmaceutical compositions described herein are in the form of syrups, capsules, and soft gels (including, for example, chewable gummies).

Techniques for formulation and administration of the compositions can be found in Remington's Pharmaceutical Sciences, 18th Ed., Mack Publishing Co, Easton, Pa., 1990. The pharmaceutical compositions described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent.

In some variations, the compositions provided herein are administered to the human as a unit dosage, for example, in the form of syrups, capsules, and soft gels (including, for example, chewable gummies) as described herein. In one variation, “unit dosage form” refers to physically discrete units, suitable as unit dosages, each unit containing a predetermined quantity of the compositions provided herein, or compositions comprising biological active(s) isolated from the compositions provided herein, which may be in a pharmaceutically acceptable carrier.

As used herein, by “pharmaceutically acceptable” refers to a material that is not biologically or otherwise undesirable, e.g., the material may be incorporated into a pharmaceutical composition administered to an individual without causing significant undesirable biological effects or interacting in a deleterious manner with any of the other components of the composition in which it is contained. Pharmaceutically acceptable carriers or excipients have preferably thus in some embodiments met the required standards of toxicological and manufacturing testing and/or are included on the Inactive Ingredient Guide prepared by the U.S. Food and Drug administration.

In other variations, the compositions provided herein are formulated for aerosol delivery.

### Dosages

In some embodiments, the methods provided comprise administering to the human in need thereof an effective amount of the compositions provided herein, including compositions comprising compounds of formulae (I), (II), (II-A), (II-B), (III), (III-A), (IV) and (IV-A). In some variations, an “effective amount” intends such amount of a composition or biological active of the invention which should be effective in a given therapeutic form. In certain variations, an effective amount of the composition provided herein is an amount sufficient to reduce eosinophil effector function in the human, and thereby treating the human suffering from the conditions, diseases or disorders described herein, or alleviating the existing symptoms of such conditions, diseases or disorders.

In some variations, exemplary dosage levels of microcolins for a human may be between 0.01 mg/kg to 100 mg/kg weight of the human.

The final dosage regimen is determined by the attending physician in view of good medical practice, considering various factors that modify the action of the salmonid oil composition, or composition comprising biological active(s) isolated from the salmonid oil compositions provided herein, the identity and severity of the disease state, the responsiveness of the subject, the age, condition, body weight, sex, and diet of the subject, and the severity of any infection. Additional factors that can be taken into account include time and frequency of administration, drug combinations, reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials.

An effective amount may be in one or more doses, i.e., a single dose or multiple doses may be required to achieve the desired treatment endpoint. In certain embodiments, the salmonid oil composition, or composition comprising biological active(s) isolated from the salmonid oil compositions provided herein, are administered once, twice, or three times daily. In certain embodiments, the composition provided herein are administered once or twice daily. In certain embodiments, the composition provided herein are administered once daily.

### Articles of Manufacture and Kits

The compositions provided herein, including compositions comprising compounds of formulae (I), (II), (II-A), (II-B), (III), (III-A), (IV) and (IV-A), may be formulated in one or more pharmaceutically acceptable carriers, excipients or other ingredients can be prepared, placed in an appropriate container, and labeled for treatment of an indicated condition, disease or disorder. Accordingly, in certain aspects, also provided is an article of manufacture, such as a container comprising a dosage form of salmonid oil composition, or composition comprising biological active(s) isolated from the compositions provided herein, and a label containing instructions for use of such compositions.

In some embodiments, the article of manufacture is a container comprising a dosage form of compositions provided herein, and one or more pharmaceutically acceptable carriers, excipients or other ingredients. In one embodiment of the articles of manufacture described herein, the dosage form is a syrup, capsule and soft gel (including, for example, chewable gummies).

In certain aspects, kits also are provided. For example, in some embodiments, a kit can comprise a dosage form of compositions provided herein, and a package insert containing instructions for use of the composition/active(s) in treatment of a condition, disease or disorder. The instructions for use in the kit may be for treating a respiratory inflammation or inflammation of the respiratory tract, including, for example, asthma. In one variation, the instructions for use in the kit may be for treating bronchial asthma. In another variation, the instructions for use in the kit may be for treating severe acute respiratory syndrome.

The labels and package inserts of the articles of manufacture and kits, respectively, contain instructions for treating any of the conditions, diseases or disorders described herein. In some embodiments, the label contain instructions for treatment of inflammatory conditions, disorders or diseases, including respiratory conditions, disorders or diseases. In some variations, the label contain instructions for treatment of a chronic inflammatory disorder of the airways. In one variation, the label contain instructions for treatment of asthma, such as bronchial asthma and/or steroid treatment resistant asthma. In other embodiments, the label contain instructions for treatment of a viral respiratory disease, such as severe acute respiratory syndrome (including, for example, severe acute respiratory syndrome caused by a coronavirus).

Unless clearly indicated otherwise, the terms “a,” “an,” and the like, refer to one or more.

In some variations, “about” refers to the usual error range for the respective value readily known to the skilled person in this technical field. Reference to “about” a value or parameter herein includes (and describes) embodiments that are directed to that value or parameter per se. For example, “about x” includes and describes “x” per se. In some embodiments, the term “about” when used in association with a measurement, or used to modify a value, a unit, a constant, or a range of values, refers to variations of +/−2%.

In some variations, “between” two values or parameters herein includes (and describes) embodiments that include those two values or parameters per se. For example, description referring to “between x and y” includes description of “x” and “y” per se.

### ENUMERATED EMBODIMENTS

The following enumerated embodiments are representative of some aspects of the invention.
1. A method for treating an inflammatory condition, disorder or disease in a human in need thereof, comprising: administering to the human an effective dose of a composition comprising at least one lipopeptide to treat the inflammatory condition, disorder or disease,
wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing; or
wherein the lipopeptide is a compound of formula (I):

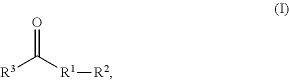

or a salt thereof, including any isomers of the foregoing, wherein:
   R¹ is at least one optionally substituted amino acid moiety;
   R² is

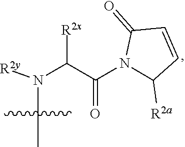

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
2. The method of embodiment 1, wherein the inflammatory condition, disorder or disease is a chronic inflammatory disorder of the airways.
3. The method of embodiment 1, wherein the inflammatory condition, disorder or disease is asthma.
4. The method of embodiment 1, wherein the inflammatory condition, disorder or disease is bronchial asthma.
5. A method for treating a respiratory condition, disorder or disease in a human in need thereof, comprising: administering to the human an effective dose of a composition comprising at least one lipopeptide to treat the inflammatory condition, disorder or disease,
wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing; or
wherein the lipopeptide is a compound of formula (I):

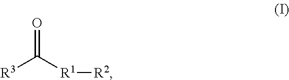

or a salt thereof, including any isomers of the foregoing, wherein:
   R¹ is at least one optionally substituted amino acid moiety;
   R² is

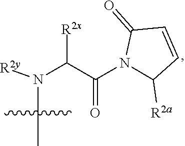

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
6. The method of embodiment 5, wherein the respiratory condition, disorder or disease is a viral respiratory disease.
7. The method of embodiment 5, wherein the respiratory condition, disorder or disease is severe acute respiratory syndrome.
8. The method of embodiment 7, wherein the severe acute respiratory syndrome is caused by a coronavirus.
9. The method of any one of embodiments 1 to 8, wherein the human is largely resistant to medical and surgical interventions for treating the condition, disorder or disease.
10. The method of any one of embodiments 1 to 4, wherein the human exhibits or has resistance to steroid treatments.
11. The method of any one of embodiments 1 to 4, wherein the human has steroid treatment resistant asthma.
12. The method of any one of embodiments 1 to 11, wherein the administration of the composition to the human reduces or delays the need to provide the human with assisted respiration.
13. A method for reducing eosinophil effector function in a human in need thereof, comprising administering to the human a salmonid oil composition or a composition comprising at least one lipopeptide to reduce eosinophil effector function,
wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing; or
wherein the lipopeptide is a compound of formula (I):

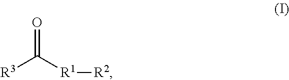

or a salt thereof, including any isomers of the foregoing, wherein:
   R¹ is at least one optionally substituted amino acid moiety;
R² is

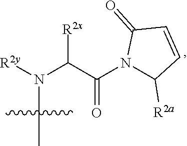

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
14. The method of any one of the preceding embodiments, wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing.
15. The method of any one of the preceding embodiments, wherein the lipopeptide is a compound of formula (I):

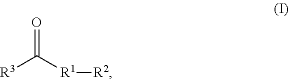

or a salt thereof, including any isomers of the foregoing, wherein:
   R¹ is at least one optionally substituted amino acid moiety;
   R² is

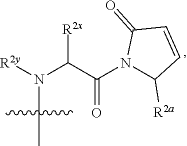

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
16. The method of embodiment 15, wherein R¹ is a sequence of three optionally substituted amino acid moieties.
17. The method of embodiment 15, wherein R¹ is —R^{1a}—R^{1b}—R^{1c}—, wherein:
R^{1a} is an optionally substituted amino acid moiety with a hydrophobic side chain;
R^{1b} is an optionally substituted amino acid moiety with a polar uncharged side chain; and
R^{1c} is an optionally substituted amino acid moiety with a hydrophobic side chain.
18. The method of embodiment 15, wherein R¹ is —R^{1a}—R^{1b}—R^{1c}—, wherein:
R^{1a} is an optionally substituted amino acid moiety selected from the group consisting of Leu, Val, and Gly;
R^{1b} is an optionally substituted amino acid moiety selected from the group consisting of Thr, Ser, and Cys; and
R^{1c} is an optionally substituted amino acid moiety selected from the group consisting of Val, Leu, and Gly.
19. The method of embodiment 15, wherein R¹ is —R^{1a}—R^{1b}—R^{1c}—, wherein:
R^{1a} is an optionally substituted amino acid moiety selected from the group consisting of Leu, Val, and Gly;
R^{1b} is a substituted amino acid moiety selected from the group consisting of Thr, Ser, and Cys, substituted with —C(O)CH₃; and
R^{1c} is an optionally substituted amino acid moiety selected from the group consisting of Val, Leu, and Gly.
20. The method of embodiment 15, wherein R¹ is:
Leu-OAcThr-Val, wherein each Leu and Val is N-methylated;
Val-OAcThr-Leu, wherein each Val and Leu is N-methylated;
Gly-OAcThr-Gly, wherein each Gly is N-methylated;
Leu-OAcSer-Val, wherein each Leu and Val is N-methylated; or
Leu-SAcCys-Val, wherein each Leu and Val is N-methylated.
21. The method of any one of embodiments 1 to 20, wherein R^{2x} and R^{2y} are taken together with the atoms to which they are attached to form an optionally substituted 5-membered heterocycle.
22. The method of any one of embodiments 1 to 20, wherein R² is:

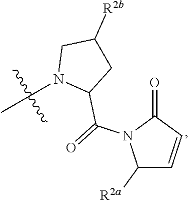

wherein R^{2b} is H or OH.
23. The method of any one of embodiments 1 to 20, wherein R² is:

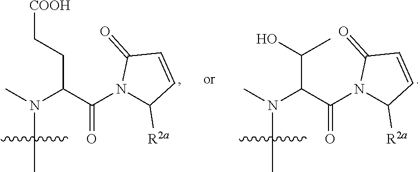

24. The method of any one of the preceding embodiments, wherein the lipopeptide is:

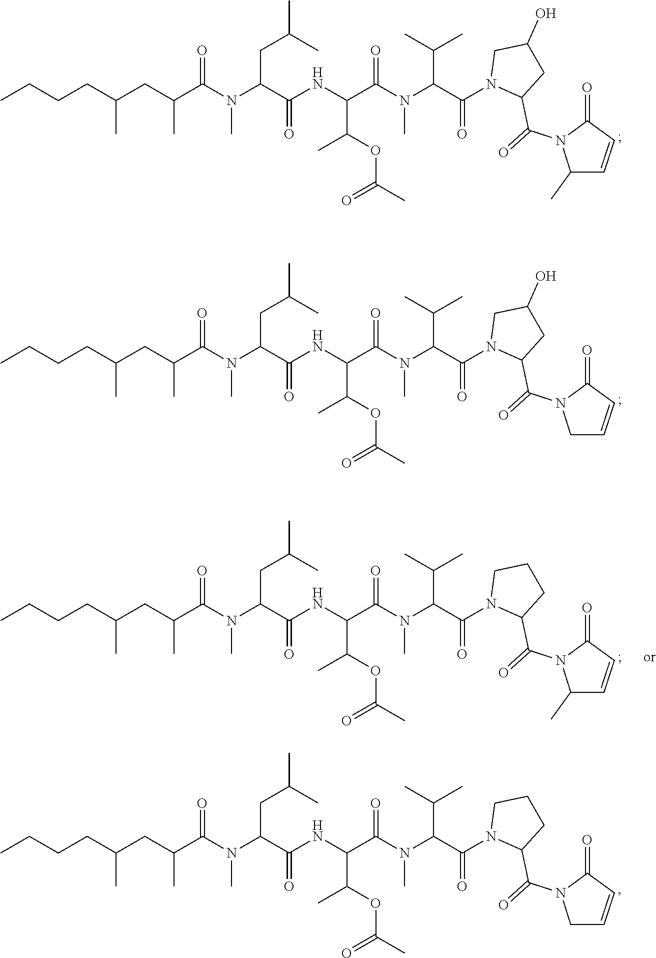 [SMILES: CCCCC(C)CC(C)C(=O)N(C)C(CC(C)C)C(=O)NC(C(=O)N(C)C(C(=O)N1CC(O)CC1C(=O)N1C(=O)C=CC1C)C(C)C)C(C)OC(C)=O ; CCCCC(C)CC(C)C(=O)N(C)C(CC(C)C)C(=O)NC(C(=O)N(C)C(C(=O)N1CC(O)CC1C(=O)N1CC=CC1=O)C(C)C)C(C)OC(C)=O ; CCCCC(C)CC(C)C(=O)N(C)C(CC(C)C)C(=O)NC(C(=O)N(C)C(C(=O)N1CCCC1C(=O)N1C(=O)C=CC1C)C(C)C)C(C)OC(C)=O ; CCCCC(C)CC(C)C(=O)N(C)C(CC(C)C)C(=O)NC(C(=O)N(C)C(C(=O)N1CCCC1C(=O)N1CC=CC1=O)C(C)C)C(C)OC(C)=O]

or a salt thereof, including any isomers of the foregoing.
25. The method of any one of the preceding embodiments, wherein the lipopeptide is:

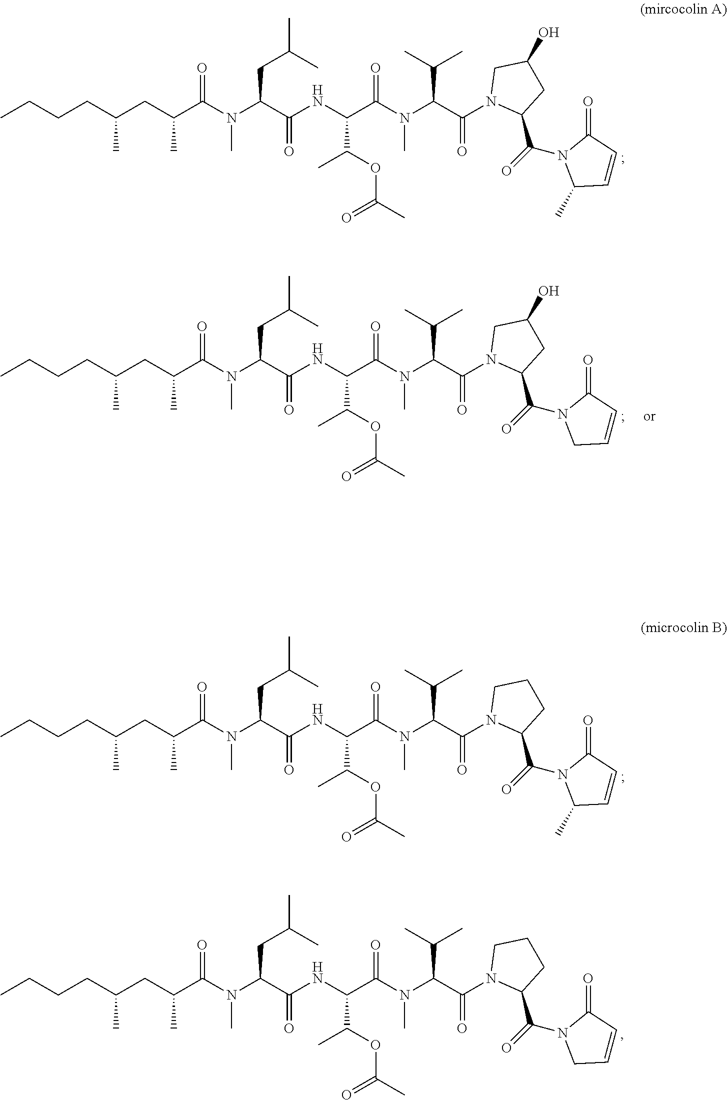

or a salt thereof.
26. The method of any one of the preceding embodiments, wherein the lipopeptide is microcolin A, or microcolin B, or a salt thereof, including any isomers of the foregoing, or any combinations of the foregoing.
27. The method of any one of the preceding embodiments, wherein the lipopeptide is microcolin.
28. The method of any one of the preceding embodiments, wherein composition further comprises caproic acid, caprylic acid, capric acid, lauric acid, behenic acid, lignoceric acid, myrstoleic acid, heptadecenoic acid, elaidic acid, gadoleic acid, erucic acid, brassidic acid, nervonic acid, gamma linolenic acid, columbinic acid, stearidonic acid, mead acid, or dihomo gamma linolenic acid, or any combination thereof.
29. The method of any one of the preceding embodiments, wherein the composition further comprises ligustilide, germacrene, thymol, eugenol, carvacrol, linalool, citronellol, terpineol, bisbalol, santalol, thujone, pinacamphone, italidone, linalyl acetate, geranyl acetate, citronellyl formate, helenalin, elecampane, furocoumarin, chavicol, sitosterol, stigmasterol, or 3-butyliden-4,5-dihydrophthalide, or any combination thereof.
30. The method of any one of the preceding embodiments, wherein the composition further comprises iturin A, hoiamides, heronamides, laxaphycin, apramides, dragonamides, gageotetrins, lyngbyabellins, cyclodycidins, parguerine, pumilacidin, sulforeido lipopeptides, fengycins, mebamamides, penicimutamides, sulfoglycolipids, halovir, kahalalide, or tuftsin, or any salt thereof, or any combination of the foregoing.
31. The method of any one of the preceding embodiments, wherein the composition is administered as a syrup, chewable, capsule or soft gel.
32. An article of manufacture, comprising:
a container comprising a composition comprising at least one lipopeptide,
   wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing; or
   wherein the lipopeptide is a compound of formula (I):

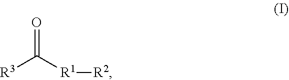

or a salt thereof, including any isomers of the foregoing, wherein:
   R¹ is at least one optionally substituted amino acid moiety;
   R² is

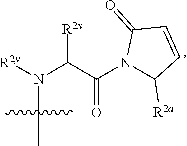

:
R^{2a} is H or optionally substituted alkyl:
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl; and
a label containing instructions for use of such composition.
33. The article of manufacture of embodiment 32, wherein the lipopeptide is a compound of formula (I):

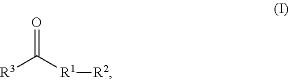

or a salt thereof, including any isomers of the foregoing, wherein:
   R¹ is at least one optionally substituted amino acid moiety;
   R² is

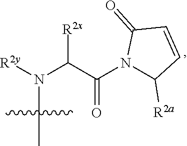

wherein:
R^{2a} is H or optionally substituted alkyl,
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
34. The article of manufacture of embodiment 32 or 33, wherein the composition is provided in a dosage form.
35. The article of manufacture of embodiment 32 or 33, wherein the dosage form is a syrup, chewable, capsule or soft gel.
36. A kit, comprising:
a dosage form of a composition comprising a composition comprising at least one lipopeptide,
   wherein the lipopeptide is [2,4-alkylsubstituted octanoyl]-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or a salt thereof, including any isomers of the foregoing; or

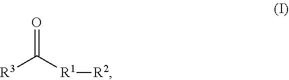

wherein the lipopeptide is a compound of formula (I):
or a salt thereof, including any isomers of the foregoing, wherein:
   R¹ is at least one optionally substituted amino acid moiety;
   R² is

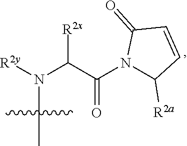

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl; and
a package insert containing instructions for use of such composition.
37. The kit of embodiment 36, wherein the lipopeptide is a compound of formula (I):

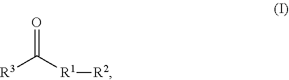

or a salt thereof, including any isomers of the foregoing, wherein:
   R¹ is at least one optionally substituted amino acid moiety;
   R² is

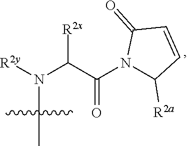

wherein:
R^{2a} is H or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkyl.
38. The kit of embodiment 36 or 37, wherein the dosage form is a syrup, chewable, capsule or soft gel.

### EXAMPLES

The following Examples are merely illustrative and are not meant to limit any aspects of the present disclosure in any way.

### SYNTHETIC EXAMPLES

Structure verification is carried out by obtaining the expected M+ ion on all final analogs synthesized. All mass resolution LC/MS data was obtained.

### Example 1

### Synthesis of Compound 1b

Step 1 (Condensation of Racemic 2,4-dimethyloctanoic acid with pTos.N-methyl-L-Leu-OBn). To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To another round-bottomed, single-necked reaction flask equipped with a micro magnetic stir bar, is added N-Methyl-L-leucine benzyl ester p-toluenesulfonate (pTos.MeN-Leu-OBn) (407 mg), followed by dichloromethane. To the resulting white suspension is added diisopropylethylamine (DIPEA) (129 mg). The suspension turns clear and this is added to the chilled CDI suspension using a syringe over 10 minutes. The suspension turns clear and stirring under a CaCl₂ drying tube is continued at room temp (RT)˜25° C. and is monitored by TLC for completion. The reaction flask is set upon a rotovap and concentrated and extracted twice with ethyl acetate, EtOAc layer is dried over MgSO₄, filtered and rotovaped to a viscous oil.

To this oil is added 3 ml of dichloromethane and stirred at RT for 10 minutes and then added 2,4-dimethyloctanoic acid (172 mg), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into an Erlenmeyer reaction flask, diluted with dichloromethane and quenched with an aqueous solution of 0.5 N HCl and extracted twice with dichloromethane, washed with NaHCO₃, dried over MgSO₄, filtered and concentrated by rotovap to give a colourless oil (371 mg) (Intermediate 1.1) LCMS shows correct M+ at 389.570; 95.1 area %.

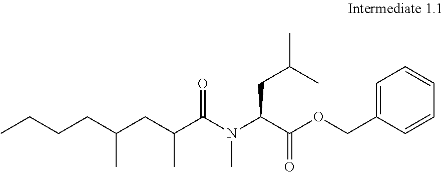 [SMILES: CCCCC(C)CC(C)C(=O)N(C)[C@@H](CC(C)C)C(=O)OCc1ccccc1]

Step 2 (Condensation of Intermediate 1.1 with OAc-L-Thr-OBn). To a round-bottomed, single-necked reaction flask, equipped with a micro magnetic stir bar, is added Intermediate 1.1 (389 mg), dry methanol and 10% Pd/C (40 mg). The reaction is stirred under H₂ atmosphere (balloon) for 2 hours, filtered and rotovaped to give a pale yellow oil (Intermediate 1.1 acid).

To a round-bottomed, single-necked reaction flask, equipped with a micro magnetic stir bar, is added N,N′-carbonyldiimidazole (CDI) (162 mg), followed by dichloromethane. Gently stirred. The reaction flask is sealed with a rubber septum containing a thermoprobe. The reaction flask is cooled to 0° C. (ice bath).

To a round-bottomed, single-necked reaction flask equipped with a micro magnetic stir bar, is added OAc-L-threonine benzyl ester hydrochloride (HCl.OAc-Thr-OBn) (287 mg), followed by dichloromethane. To the resulting white suspension is added diisopropylethylamine (DIPEA) (129 mg). The suspension turns clear and this is added to the chilled CDI suspension using a syringe over 10 minutes. The suspension turns clear and stirring under a CaCl₂ drying tube is continued for 20 hours at room temp. The reaction is monitored by TLC for completion. The reaction flask is set upon a rotovap and concentrated and extracted 2× with ethyl acetate, EtOAc layer is dried over MgSO₄, filtered and rotovaped to a viscous oil.

To the oil is added dichloromethane and stirred at RT for 10 minutes and then added Intermediate 1.1 acid (from above), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a Erlenmeyer reaction flask, diluted with dichloromethane and quenched with an aqueous solution of 0.5 N HCl and extracted twice with dichloromethane, washed with NaHCO₃, dried over MgSO₄, filtered and concentrated by rotovap to give a pale yellow oil (452 mg) (Intermediate 1.2) LCMS shows correct M+ at 532.711; 90.7 area %.

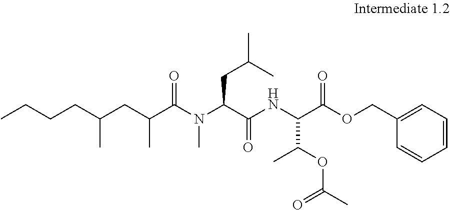 [SMILES: CCCCC(C)CC(C)C(=O)N(C)[C@@H](CC(C)C)C(=O)N[C@H](C(=O)OCc1ccccc1)C(C)OC(C)=O]

Step 3 (Condensation of Intermediate 1.2 with MeN-L-Val-OBn). To a round-bottomed, single-necked reaction flask, equipped with a micro magnetic stir bar, is added Intermediate 1.2 (532 mg), dry methanol and 10% Pd/C (40 mg). The reaction is stirred under H₂ atmosphere (balloon) for 2 hours, filtered and rotovaped to give a pale yellow oil (Intermediate 1.2 acid).

To a round-bottomed, single-necked reaction flask, equipped with a micro magnetic stir bar, is added N,N′-carbonyldiimidazole (CDI) (162 mg), followed by of dichloromethane. Gently stirred. The reaction flask is sealed with a rubber septum containing a thermoprobe. The reaction flask is cooled to 0° C. (ice bath).

To around-bottomed, single-necked reaction flask equipped with a micro magnetic stir bar, is added N-Methyl-L-valine benzyl ester 4-toluenesulfonate (pTos.MeN-L-Val-OBn) CAS #42492-62-6 (393 mg), followed by dichloromethane. To the resulting suspension is added diisopropylethylamine (DIPEA) (129 mg). The suspension turns clear and this is added to the chilled CDI suspension reaction flask using a syringe over 10 minutes. The suspension turns clear/colourless and stirring under a CaCl₂ drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added dichloromethane and stirred at RT for 10 minutes and then added Intermediate 1.2 acid (from above), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into an Erlenmeyer reaction flask, diluted with dichloromethane (8 ml) and quenched with an aqueous solution of 0.5 N HCl and extracted twice with dichloromethane, washed with NaHCO₃, dried over MgSO₄, filtered and concentrated by rotovap to give a pale yellow oil (568 mg) (Intermediate 1.3) LCMS shows correct M+ at 645.869; 91.0 area %.

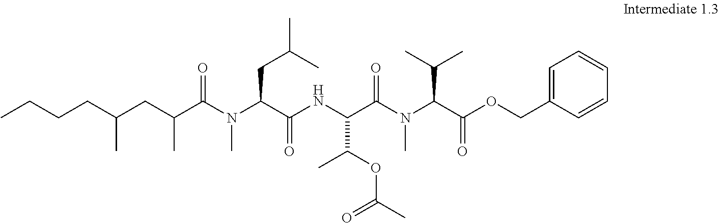 [SMILES: CCCCC(C)CC(C)C(=O)N(C)[C@@H](CC(C)C)C(=O)N[C@H](C(=O)N(C)[C@H](C(=O)OCc1ccccc1)C(C)C)C(C)OC(C)=O]

Step 4 (Condensation of Intermediate 1.3 with cis-hydroxyproline 4-methylpyrrolidinone amide). To a round-bottomed, single-necked reaction flask, equipped with a micro magnetic stir bar, is added Intermediate 1.3 (645 mg), dry methanol and 10% Pd/C (40 mg). The reaction is stirred under H₂ atmosphere (balloon) for 2 hours, filtered and rotovaped to give a pale yellow oil (Intermediate 1.3 acid).

To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a 20 ml round-bottomed, single-necked reaction flask equipped with a micro magnetic stir bar, is added cis-hydroxyproline 4-methylpyrrolidinone amide (210 mg), followed by dichloromethane (10 ml). To this is added diisopropylethylamine (DIPEA) (129 mg) and this is added to the chilled CDI suspension reaction flask using a syringe over 10 minutes. The suspension is stirred under a CaCl₂) drying tube for 20 hours at room temp (RT) ˜25° C. The reaction is monitored by TLC for completion. The reaction flask is set upon a rotovap and concentrated and extracted twice . . . ethyl acetate, EtOAc layer is dried over MgSO₄, filtered and rotovaped to a viscous oil.

To the oil is added of dichloromethane and stirred at RT for 10 minutes and then added Intermediate 1.3 acid (from above), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane and quenched with an aqueous solution of 0.5 N HCl and extracted twice with dichloromethane, washed with NaHCO₃, dried over MgSO₄, filtered and concentrated by rotovap to give a pale yellow oil (401 mg) (Compound 1b) LCMS shows correct M+ at 747.478; 78.4 area %—same as the authentic Microcolin A sample.

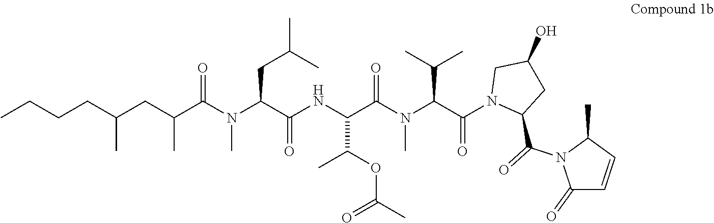 [SMILES: CCCCC(C)CC(C)C(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O]

### Example 2

### Synthesis of Compound 5a

Step 1 (Condensation of Octanoic acid with pTos.N-methyl-L-Leu-OBn). To a round-bottomed, single-necked reaction flask, equipped with a micro magnetic stir bar, is added N,N′-carbonyldiimidazole (CDI) (162 mg), followed by dichloromethane. Gently stirred. The reaction flask is sealed with a rubber septum containing a thermoprobe. The reaction flask is cooled to 0° C. (ice bath).

To a round-bottomed, single-necked reaction flask equipped with a micro magnetic stir bar, is added N-Methyl-L-leucine benzyl ester p-toluenesulfonate (PTS.HMeN-Leu-OBn) (407 mg), followed by dichloromethane. To the resulting suspension is added diisopropylethylamine (DIPEA) (129 mg). The suspension turns clear and this is added to the chilled CDI suspension reaction flask using a syringe over 10 minutes. The suspension turns clear and stirring under a CaCl₂) drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added 3 ml of dichloromethane and stirred at RT for 10 minutes and then is added Octanoic acid (144 mg), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane and quenched with an aqueous solution of 0.5 N HCl and extracted twice with dichloromethane, washed with NaHCO₃, dried over MgSO₄, filtered and concentrated by rotovap to give a colourless oil (380 mg) (Intermediate 5.1) LCMS shows correct M+ at 361.517; 96.4 area %.

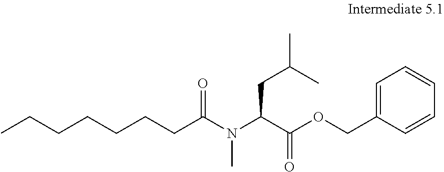 [SMILES: CCCCCCCC(=O)N(C)[C@@H](CC(C)C)C(=O)OCc1ccccc1]

Intermediate 5.1 is converted to Compound 5b using the same Step 2, Step 3 and Step 4 as shown in Example 1 to yield 413 mg of a yellow oil (Compound 5b) LCMS shows correct M+ at 719.908; 81.7 area %.

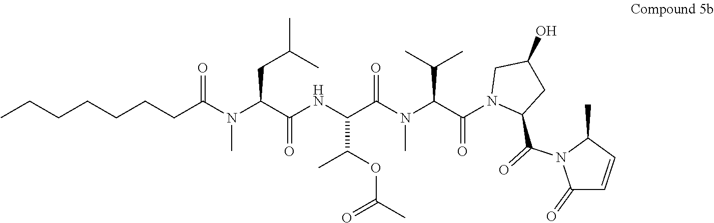 [SMILES: CCCCCCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O]

### Example 3

### Synthesis of Compound 6a

Step 1 (Condensation of Racemic 2,4-dimethyloctanoic acid with L-Leu-Obn. To a round-bottomed, single-necked reaction flask, equipped with a micro magnetic stir bar, is added N,N′-carbonyldiimidazole (CDI) (162 mg), followed by of dichloromethane. Gently stirred. The reaction flask is sealed with a rubber septum containing a thermoprobe. The reaction flask is cooled to 0° C. (ice bath).

To a round-bottomed, single-necked reaction flask equipped with a micro magnetic stir bar, is added N-Methyl-L-leucine benzyl ester p-toluenesulfonate (L-Leu-OBn) (221 mg), followed by dichloromethane. To the resulting suspension is added diisopropylethylamine (DIPEA) (129 mg). The suspension turns clear and this is added to the chilled CDI suspension reaction flask using a syringe over 10 minutes. The suspension turns clear and stirring under a CaCl₂ drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added dichloromethane and stirred at RT for 10 minutes and then added 2,4-dimethyloctanoic acid (172 mg), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a Erlenmeyer reaction flask, diluted with dichloromethane and quenched with an aqueous solution of 0.5 N HCl and extracted twice with dichloromethane, washed with NaHCO₃, dried over MgSO₄, filtered and concentrated by rotovap to give a colourless oil (333 mg) (Intermediate 6.1) LCMS shows correct M+ at 375.544; 96.6 area %.

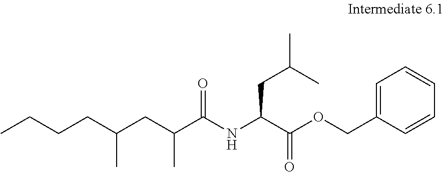 [SMILES: CCCCC(C)CC(C)C(=O)N[C@@H](CC(C)C)C(=O)OCc1ccccc1]

Step 2. Intermediate 6.1 is condensed with OAc-L-threonine benzyl ester hydrochloride in exactly the same manner as shown in Step 2 Example 1 to yield Intermediate 6.2 (407 mg) LCMS shows correct M+ at 518.684; 95.2 area %.

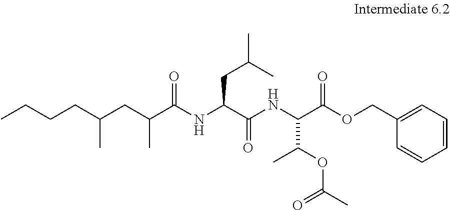 [SMILES: CCCCC(C)CC(C)C(=O)N[C@@H](CC(C)C)C(=O)N[C@H](C(=O)OCc1ccccc1)C(C)OC(C)=O]

Steps 3-4. Intermediate 6.2 is first condensed with L-valine benzyl ester 4-toluenesulfonate in the same manner as described in Step 3 Example 1 and the resultant product is condensed with cis-hydroxyproline 4-methylpyrrolidinone amide in the same manner as shown in Step 4 Example 1 to yield Compound 6a (412 mg) as a pale yellow oil. LCMS shows correct M+ at 719.907; 88.1 area %.

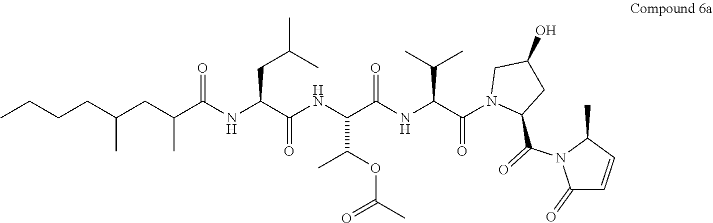 [SMILES: CCCCC(C)CC(C)C(=O)N[C@@H](CC(C)C)C(=O)N[C@H](C(=O)N[C@H](C(=O)N1C[C@@H](O)C[C@H]1C(=O)N1C(=O)C=C[C@@H]1C)C(C)C)C(C)OC(C)=O]

### Example 4

### Synthesis of Compound 7a

2,4-dimethyloctanoic acid is condensed with N-methyl-L-glycine benzyl ester·HCl in the same manner as shown in Step 1 Example 1 to yield

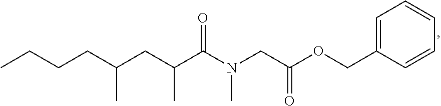 [SMILES: CCCCC(C)CC(C)C(=O)N(C)CC(=O)OCc1ccccc1]

which is further condensed with OAc-L-threonine benzyl ester hydrochloride as shown in Step 2 Example 1 to yield

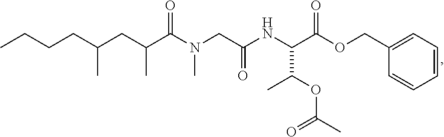 [SMILES: CCCCC(C)CC(C)C(=O)N(C)CC(=O)N[C@H](C(=O)OCc1ccccc1)C(C)OC(C)=O]

which is further condensed with N-methyl-L-glycine benzyl ester. HCl as shown in Step 3 Example 1 to yield

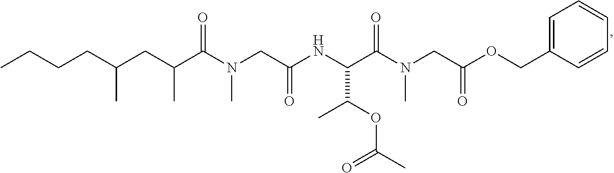 [SMILES: CCCCC(C)CC(C)C(=O)N(C)CC(=O)N[C@H](C(=O)N(C)CC(=O)OCc1ccccc1)C(C)OC(C)=O]

which is finally condensed with cis-hydroxyproline 4-methylpyrrolidinone amide as shown in Step 4 Example 1 to yield Compound 7a (357 mg) LCMS shows correct M+ at 649.775, 86.4 area %.

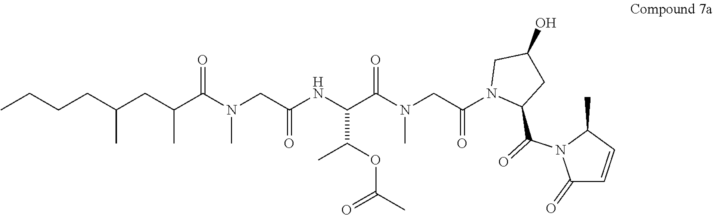 [SMILES: CCCCC(C)CC(C)C(=O)N(C)CC(=O)N[C@H](C(=O)N(C)CC(=O)N1C[C@@H](O)C[C@H]1C(=O)N1C(=O)C=C[C@@H]1C)C(C)OC(C)=O]

### Example 5

### Synthesis of Compound 8a

Intermediate 1.1 is condensed with L-threonine benzyl ester hydrochloride by the process similar to that shown in Step 2 Example 1 to yield

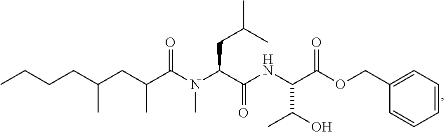 [SMILES: CCCCC(C)CC(C)C(=O)N(C)[C@@H](CC(C)C)C(=O)N[C@H](C(=O)OCc1ccccc1)C(C)O]

which is further condensed with N-Methyl-L-valine benzyl ester 4-toluenesulfonate in the same manner as described in Step 3 Example 1 and the resultant product is condensed with cis-hydroxyproline 4-methylpyrrolidinone amide in the same manner as shown in Step 4 Example 1 to yield Compound 8a (521 mg) as a yellow oil. LCMS shows correct M+ at 705.924; 79.5 area %.

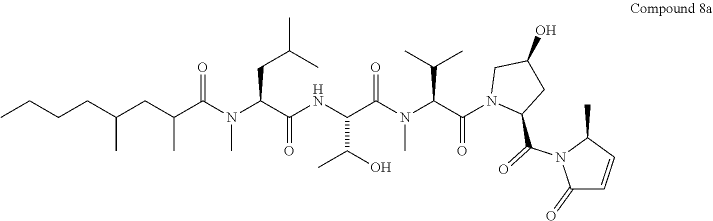 [SMILES: CCCCC(C)CC(C)C(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O]

### Example 6

### Synthesis of Compound 9a

Intermediate 1.1 is condensed with OAc-L-serine benzyl ester hydrochloride using the process as shown in Step 2 Example 1 to yield

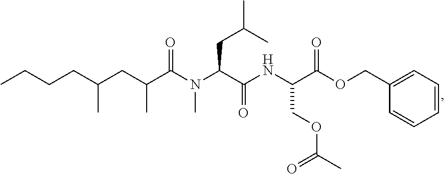 [SMILES: CCCCC(C)CC(C)C(=O)N(C)[C@@H](CC(C)C)C(=O)N[C@@H](COC(C)=O)C(=O)OCc1ccccc1]

which is further condensed with N-Methyl-L-valine benzyl ester 4-toluenesulfonate in the same manner as described in Step 3 Example 1 and the resultant product is condensed with cis-hydroxyproline 4-methylpyrrolidinone amide in the same manner as shown in Step 4 Example 1 to yield Compound 9a (668 mg) as a colourless oil. LCMS shows correct M+ at 733.934; 82.4 area %.

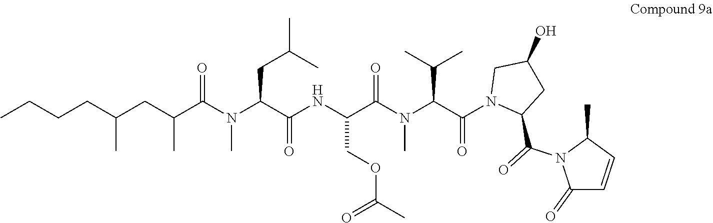 [SMILES: CCCCC(C)CC(C)C(=O)N(C)[C@@H](CC(C)C)C(=O)N[C@@H](COC(C)=O)C(=O)N(C)[C@H](C(=O)N1C[C@@H](O)C[C@H]1C(=O)N1C(=O)C=C[C@@H]1C)C(C)C]

### Example 7

### Synthesis of Compound 10a

Steps 1-3. Steps 1, 2 and 3 were carried out as in Compound 1b synthesis to yield Intermediate 1.3 which is further condensed with 3-Hydroxy-2-(methylamino)-1-(5-methyl-2-oxo-3-cyclopenten-1-yl)-1-propanone as show in Step 4 below.

Step 4. To a round-bottomed, single-necked reaction flask, equipped with a micro magnetic stir bar, is added Intermediate 1.3 (645 mg), dry methanol and 10% Pd/C (40 mg). The reaction is stirred under H₂ atmosphere (balloon) for 2 hours, filtered and rotovaped to give a pale yellow oil (Intermediate 1.3 acid).

To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a 20 ml round-bottomed, single-necked reaction flask equipped with a micro magnetic stir bar, is added 3-Hydroxy-2-(methylamino)-1-(5-methyl-2-oxo-3-cyclopenten-1-yl)-1-propanone (197 mg), followed by dichloromethane (10 ml). To this is added diisopropylethylamine (DIPEA) (129 mg) and this is added to the chilled CDI suspension using a syringe over 10 minutes. The suspension is stirred under a CaCl₂) drying tube for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a yellow oil.

To the oil is added of dichloromethane and stirred at RT for 10 minutes and then added Intermediate 1.3 acid (from above), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane (8 ml) and quenched with an aqueous solution of 0.5 N HCl (10 ml) and extracted twice with 10 ml dichloromethane, washed with NaHCO₃, dried over MgSO₄, filtered and concentrated by rotovap to give a light brown oil Compound 10a (514 mg). LCMS shows correct M+ at 721.923, 75.8 area %.

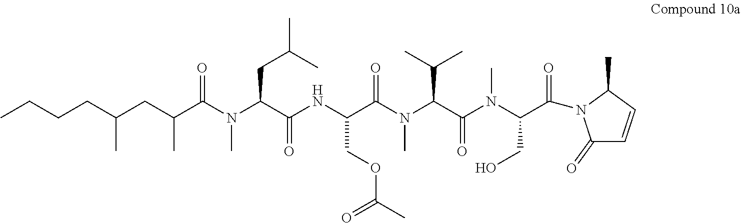 [SMILES: CCCCC(C)CC(C)C(=O)N(C)[C@@H](CC(C)C)C(=O)N[C@@H](COC(C)=O)C(=O)N(C)[C@H](C(=O)N(C)[C@@H](CO)C(=O)N1C(=O)C=C[C@@H]1C)C(C)C]

### Example 8

### Synthesis of Compound 11a

Intermediate 1.1 is condensed with L-cysteine benzyl ester hydrochloride using a similar process to that shown in Step 2 Example 1 to yield

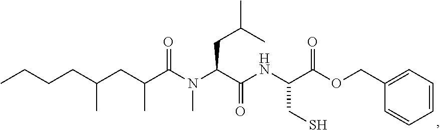 [SMILES: CCCCC(C)CC(C)C(=O)N(C)[C@@H](CC(C)C)C(=O)N[C@@H](CS)C(=O)OCc1ccccc1]

which is further condensed with N-Methyl-L-valine benzyl ester 4-toluenesulfonate in the same manner as described in Step 3 Example 1 and the resultant product is condensed with L-proline 4-methylpyrrolidinone amide (194 mg) in the same manner as shown in Step 4 Example 1 to yield Compound 11a (439 mg) as a yellow oil. LCMS shows correct M+ at 691.967; 72.1 area %.

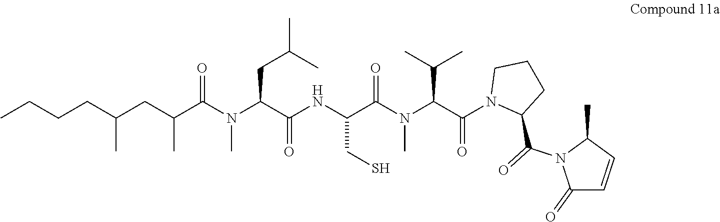 [SMILES: CCCCC(C)CC(C)C(=O)N(C)[C@@H](CC(C)C)C(=O)N[C@@H](CS)C(=O)N(C)[C@H](C(=O)N1CCC[C@H]1C(=O)N1C(=O)C=C[C@@H]1C)C(C)C]

### Example 9

### Synthesis of Compound 12a

Intermediate 1.3 is condensed with L-proline-maleimide (194 mg) in the same manner as shown in Step 4 Example 1 to yield Compound 12a (539 mg) as a brown oil. LCMS shows correct M+ at 731.918; 77.8 area %.

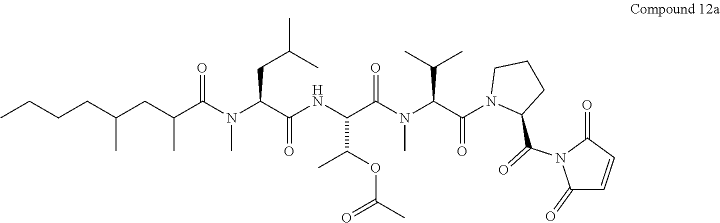 [SMILES: CCCCC(C)CC(C)C(=O)N(C)[C@@H](CC(C)C)C(=O)N[C@H](C(=O)N(C)[C@H](C(=O)N1CCC[C@H]1C(=O)N1C(=O)C=CC1=O)C(C)C)C(C)OC(C)=O]

### Example 10

### Synthesis of Compound 13a

Step 1 (Condensation of cyclohexane carboxylic acid with N-methyl-L-Leu-OBn). To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a CDI round-bottomed, single-necked reaction flask equipped with a micro magnetic stir bar, is added N-Methyl-L-leucine benzyl ester p-toluenesulfonate (PTS.HMeN-Leu-OBn) (407 mg), followed by dichloromethane (2 ml). To the resulting suspension is added diisopropylethylamine (DIPEA) (129 mg). The suspension turns clear and this is added to the chilled CDI suspension reaction flask using a syringe over 10 minutes. The suspension turns clear and stirring under a CaCl₂) drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added 3 ml of dichloromethane and stirred at RT for 10 minutes and then is added cyclohexane carboxylic acid (128 mg), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane (7 ml) and quenched with an aqueous solution of 0.5 N HCl (10 ml) and extracted twice with 10 ml dichloromethane, washed with NaHCO₃, dried over MgSO₄, filtered and concentrated by rotovap to yield a yellow oil (LCMS shows correct M+ at 345.475; 94.2. area %)

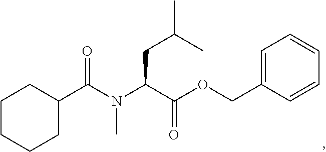 [SMILES: CC(C)C[C@@H](C(=O)OCc1ccccc1)N(C)C(=O)C1CCCCC1]

which is condensed with L-Thr-OBn in a process similar to Step 2 Example 1, followed by condensation with N-Methyl-L-Valine.OBn as in Step 3 Example 1 and finally condensed with cis-hydroxyproline 4-methylpyrrolidinone amide as in Step 4 Example 1 to yield 413 mg of a pale brown oil Compound 13a—LCMS shows correct M+ at 663.801; 82.7 area %.

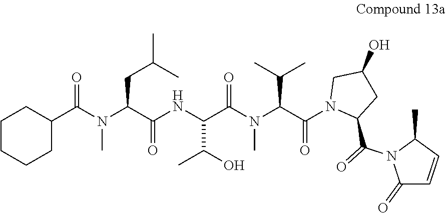 [SMILES: CC(C)C[C@@H](C(=O)N[C@H](C(=O)N(C)[C@H](C(=O)N1C[C@@H](O)C[C@H]1C(=O)N1C(=O)C=C[C@@H]1C)C(C)C)C(C)O)N(C)C(=O)C1CCCCC1]

### Example 11

### Synthesis of Compound 14a

EPA acid (CAS No. 10417-94-4) (302 mg) is condensed with N-methyl-L-Leu-OBn in the same manner as shown in Compound 13a to yield a brown oil (LCMS shows correct M+at 519.756; 92.5. area %)

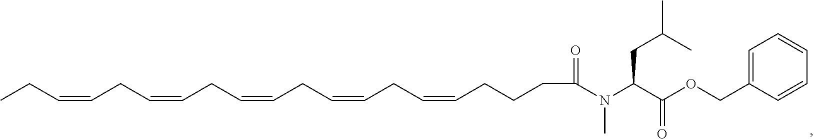 [SMILES: CC/C=C\C/C=C\C/C=C\C/C=C\C/C=C\CCCC(=O)N(C)[C@@H](CC(C)C)C(=O)OCc1ccccc1]

which is condensed with L-Thr-OBn in a process similar to Step 2 Example 1, followed by condensation with N-Methyl-L-Valine. OBn as in Step 3 Example 1 and finally condensed with cis-hydroxyproline 4-methylpyrrolidinone amide as in Step 4 Example 1 to yield 684 mg of a brown viscous oil Compound 14a—LCMS shows correct M+ at 838.083; 77.0 area %.

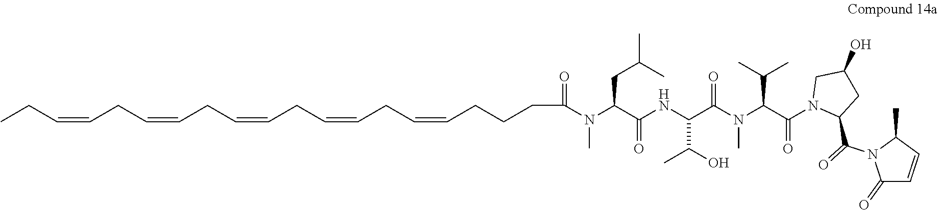 [SMILES: CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O]

### Example 12

### Synthesis of Compound 15a

DHA acid (CAS Number 6217-54-5) (328 mg) is condensed with N-methyl-L-Leu-OBn in the same manner as shown in Example 1 to yield a brown waxy solid (LCMS shows correct M+ at 545.793; 90.6 area %)

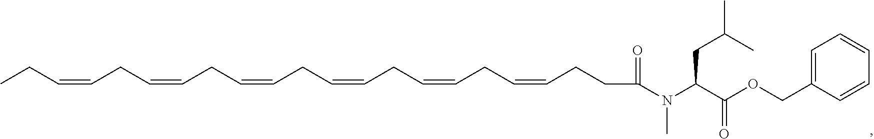 [SMILES: CC/C=C\C/C=C\C/C=C\C/C=C\C/C=C\C/C=C\CCC(=O)N(C)[C@@H](CC(C)C)C(=O)OCc1ccccc1]

which is condensed with L-Thr-OBn in a process similar to Step 2 Example 1, followed by condensation with N-Methyl-L-Valine.OBn as in Step 3 Example 1 and finally condensed with cis-hydroxyproline 4-methylpyrrolidinone amide as in Step 4 Example 1 to yield 660 mg of a brown solid Compound 15a—LCMS shows correct M+ at 864.120; 75.3 area %.

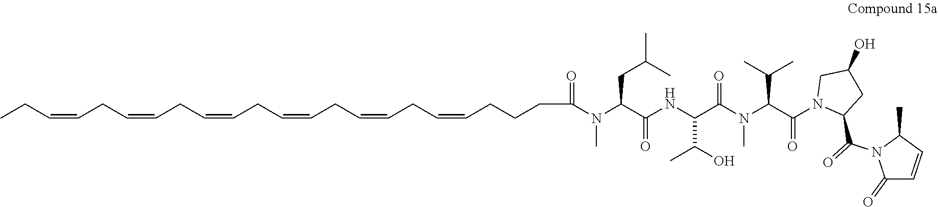 [SMILES: CC/C=C\C/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)O]

### Example 13

### Synthesis of Compound 16a

Steps 1-3. Steps 1, 2 and 3 were carried out exactly as in the Synthesis of Compound 5a to yield a yellow oil (562 mg) Intermediate 16.3 as shown below,

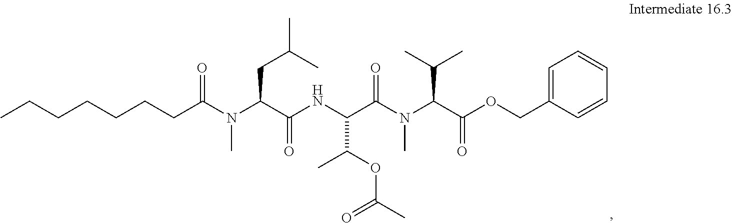 [SMILES: CCCCCCCC(=O)N(C)[C@@H](CC(C)C)C(=O)N[C@H](C(=O)N(C)[C@H](C(=O)OCc1ccccc1)C(C)C)C(C)OC(C)=O]

which is condensed with 1-{[(2S,4S)-4-Hydroxy-2-pyrrolidinyl]carbonyl}-2-piperidinone (212 mg) in the same manner as shown in Step 4 Example 1 to yield a yellow waxy solid (504 mg) Compound 16a. (LCMS shows correct M+ at 707.897; 91.8 area %).

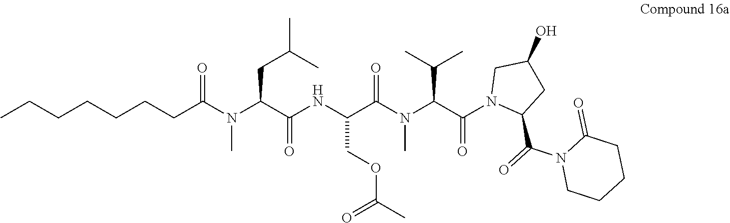 [SMILES: CCCCCCCC(=O)N(C)[C@@H](CC(C)C)C(=O)N[C@@H](COC(C)=O)C(=O)N(C)[C@H](C(=O)N1C[C@@H](O)C[C@H]1C(=O)N1CCCCC1=O)C(C)C]

### Example 14

### Synthesis of Compound 17a

Intermediate 16.3 is condensed with OMe-L-hydroxyproline (145 mg) in the same manner as shown in Step 4 Example 1 to yield a brown viscous oil (504 mg) Compound 17a. (LCMS shows correct M+ at 640.808; 84.3 area %).

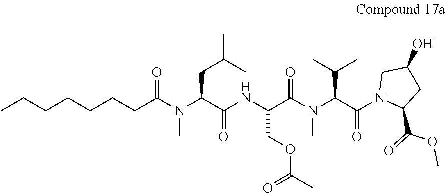 [SMILES: CCCCCCCC(=O)N(C)[C@@H](CC(C)C)C(=O)N[C@@H](COC(C)=O)C(=O)N(C)[C@H](C(=O)N1C[C@@H](O)C[C@H]1C(=O)OC)C(C)C]

### Example 15

### Synthesis of Compound 18a

Step 1 (Condensation of Octanoic acid with N-methyl-L-Phe-OBn). To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a CDI round-bottomed, single-necked reaction flask equipped with a micro magnetic stir bar, is added N-Methyl-L-phenylalanine benzyl ester (MeN-Phe-OBn) (269 mg), followed by dichloromethane (2 ml). To the resulting suspension is added diisopropylethylamine (DIPEA) (129 mg). This solution is added to the chilled CDI suspension reaction flask using a syringe over 10 minutes. Stirring under a CaCl₂ drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added 3 ml of dichloromethane and stirred at RT for 10 minutes and then is added octanoic acid (144 mg), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane (7 ml) and quenched with an aqueous solution of 0.5 N HCl (10 ml) and extracted twice with 10 ml dichloromethane, washed with NaHCO₃, dried over MgSO₄, filtered and concentrated by rotovap to give a colourless oil (406 mg) (Intermediate 18.1) LCMS shows correct M+ at 395.533; 93.9. area %.

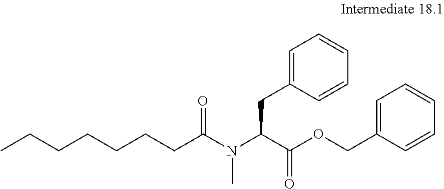 [SMILES: CCCCCCCC(=O)N(C)[C@@H](Cc1ccccc1)C(=O)OCc1ccccc1]

The above compound is sequentially condensed with OAc-L-Thr.OBn, followed by condensation with N-Methyl-L-valine benzyl ester 4-toluenesulfonate to yield

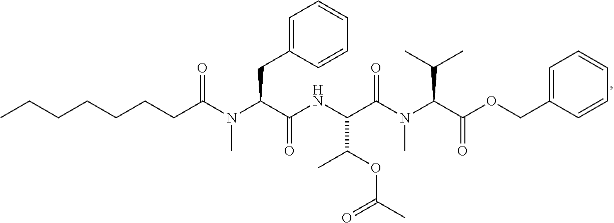 [SMILES: CCCCCCCC(=O)N(C)[C@@H](Cc1ccccc1)C(=O)N[C@H](C(=O)N(C)[C@H](C(=O)OCc1ccccc1)C(C)C)C(C)OC(C)=O]

which is finally condensed with L-hydroxyproline-maleimide (210 mg) in the same manner as shown in Step 4 Example 1 to yield Compound 18a (551 mg) as a yellow oil. LCMS shows correct M+ at 753.880; 78.4 area %.

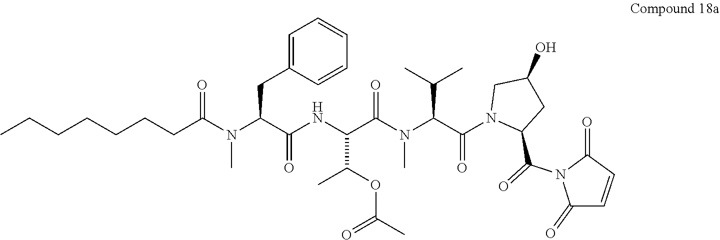 [SMILES: CCCCCCCC(=O)N(C)[C@@H](Cc1ccccc1)C(=O)N[C@H](C(=O)N(C)[C@H](C(=O)N1C[C@@H](O)C[C@H]1C(=O)N1C(=O)C=CC1=O)C(C)C)C(C)OC(C)=O]

### Example 16

### Synthesis of Compound 19a

Steps 1 and 2 from Example 2 were repeated to yield

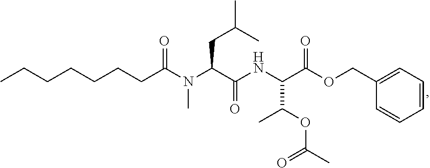 [SMILES: CCCCCCCC(=O)N(C)[C@@H](CC(C)C)C(=O)N[C@H](C(=O)OCc1ccccc1)C(C)OC(C)=O]

which is condensed with N-Methyl-L-methionine benzyl ester in a similar manner as shown in Step 3 Example 1 and then condensed with L-hydroxyproline-maleimide (210 mg) in the same manner as shown in Step 4 to yield Compound 19a (594 mg) as a brown oil. LCMS shows correct M+ at 751.932; 78.4 area %.

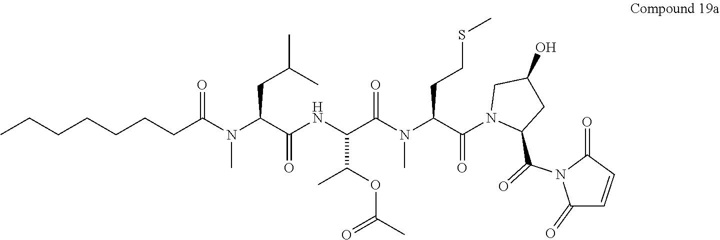 [SMILES: CCCCCCCC(=O)N(C)[C@@H](CC(C)C)C(=O)N[C@H](C(=O)N(C)[C@@H](CCSC)C(=O)N1C[C@@H](O)C[C@H]1C(=O)N1C(=O)C=CC1=O)C(C)OC(C)=O]

### Example 17

### Synthesis of Compound 20a

Step 1 (Condensation of Octanoic acid with pTos.N-methyl-L-Ile-OBn). To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a CDI round-bottomed, single-necked reaction flask equipped with a micro magnetic stir bar, is added N-Methyl-L-Isoleucine benzyl ester p-toluenesulfonate CAS #201544-39-0 (pTos.MeN-Ile-OBn) (407 mg), followed by dichloromethane (2 ml). To the resulting suspension is added diisopropylethylamine (DIPEA) (129 mg). The suspension turns clear and this is added to the chilled CDI suspension reaction flask using a syringe over 10 minutes. The suspension turns clear and stirring under a CaCl₂) drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added 3 ml of dichloromethane and stirred at RT for 20 minutes and then is added Octanoic acid (144 mg), 1-hydroxybenzotriazole hydrate (HOBt hydrate) (2 mg) and CuBr₂ (10 mg). The reaction flask is closed with a rubber septum and stirred at RT for 36 h. Upon completion of the reaction (TLC), the mixture is transferred into a 25 ml Erlenmeyer reaction flask, diluted with dichloromethane (7 ml) and quenched with an aqueous solution of 0.5 N HCl (10 ml) and extracted twice with 10 ml dichloromethane, washed with NaHCO₃, dried over MgSO₄, filtered and concentrated by rotovap to give a pale yellow oil

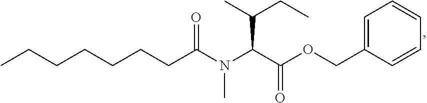 [SMILES: CCCCCCCC(=O)N(C)[C@H](C(=O)OCc1ccccc1)C(C)CC]

which is condensed with OAc-L-Thr-OBn as shown in Step 2 Example 1 to yield as a yellow oil

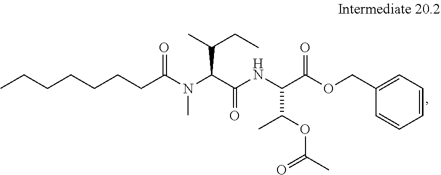 [SMILES: CCCCCCCC(=O)N(C)[C@H](C(=O)N[C@H](C(=O)OCc1ccccc1)C(C)OC(C)=O)C(C)CC]

which is condensed with N-Methyl-L-Tryptophan benzyl ester: To a round-bottomed, single-necked reaction flask, equipped with a micro magnetic stir bar, is added 532 mg of Step-2-MA-017, dry methanol and 10% Pd/C (40 mg). The reaction is stirred under H₂ atmosphere (balloon) for 2 hours, filtered and rotovaped to give a pale yellow oil (Intermediate 20.2 acid).

To a round-bottomed, single-necked reaction flask, equipped with a micro magnetic stir bar, is added N,N′-carbonyldiimidazole (CDI) (162 mg), followed by 1 ml of dichloromethane. Gently stirred. The reaction flask is sealed with a rubber septum containing a thermoprobe. The reaction flask is cooled to 0° C. (ice bath).

To a 10 ml round-bottomed, single-necked reaction flask equipped with a micro magnetic stir bar, is added N-Methyl-L-tryptophan benzyl ester·HCl (N-Me-L-Trp-OBn·HCl) (344 mg), followed by dichloromethane (2 ml). To the resulting suspension is added diisopropylethylamine (DIPEA) (129 mg). The suspension turns clear and this is added to the chilled CDI suspension reaction flask using a syringe over 10 minutes. The suspension turns clear/colourless and stirring under a CaCl₂) drying tube is continued for 20 hours at room temp. The reaction is monitored by TLC for completion. The reaction flask is set upon a rotovap and concentrated and extracted twice with ethyl acetate, EtOAc layer is dried over MgSO₄, filtered and rotovaped to a viscous oil.

To the oil is added of dichloromethane and stirred at RT for 10 minutes and then added Intermediate 20.2 (from above), 1-hydroxybenzotriazole hydrate (HOBt hydrate) (2 mg) and CuBr₂ (10 mg). The reaction flask is closed with a rubber septum and stirred at RT for 24 h. Upon completion of the reaction (TLC), the mixture is transferred into a 25 ml Erlenmeyer reaction flask, diluted with dichloromethane (8 ml) and quenched with an aqueous solution of 0.5 N HCl (10 ml) and extracted twice with 10 ml dichloromethane, washed with NaHCO₃, dried over MgSO₄, filtered and concentrated by rotovap to give a pale yellow oil (485 mg) (intermediate 20.3) LCMS shows correct M+ at 704.894; 77.2 area %,

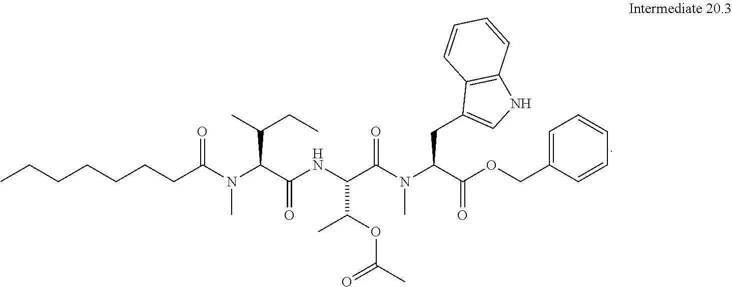 [SMILES: CCCCCCCC(=O)N(C)[C@H](C(=O)N[C@H](C(=O)N(C)[C@@H](Cc1c[nH]c2ccccc12)C(=O)OCc1ccccc1)C(C)OC(C)=O)C(C)CC]

which is condensed with L-hydroxyproline-maleimide (210 mg) in the same manner as shown in Step 4 Example 1 to yield Compound 20a (594 mg) as a pale yellow oil. LCMS shows correct M+ at 806.943; 78.4 area %

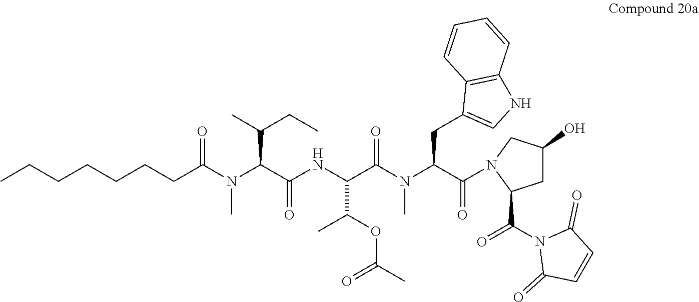 [SMILES: CCCCCCCC(=O)N(C)[C@H](C(=O)N[C@H](C(=O)N(C)[C@@H](Cc1c[nH]c2ccccc12)C(=O)N1C[C@@H](O)C[C@H]1C(=O)N1C(=O)C=CC1=O)C(C)OC(C)=O)C(C)CC]

### BIOLOGICAL EXAMPLES

### Example 18

### Percent Eosinophil Apoptosis in Allergic Human Purified Peripheral Blood Eosinophils

In triplicate, Allergic Human Eosinophils were placed in RPMI 1640 medium supplemented with IL-5 (50 pM), 1% FBS and PenStrep in the presence of 3 μg/mL ApoA-IV (positive control), 10 μg/mL test compounds, and formulation vehicle (negative control). Aliquots were removed after 18 hr incubation, washed twice in PBS, and resuspended in binding buffer. The eosinophil cells were stained using the Annexin V-FITC Apoptosis Detection Kit I, (Sigma Aldrich) and immediately analyzed by flow cytometry. Each sample was acquired for 1 min, and the total number of eosinophils gated on a forward scatter/side scatter plot and the percentage of live cells (annexin Vneg) and apoptotic cells (annexin Vpos) was recorded. (Standard deviation in the assay is +/−2.3%). See results in Table 1 below.

TABLE 1

| Compound | Percent live | Percent of | Total percent live and |
|---|---|---|---|
| No. | EO cells | apoptotic EO cells | apoptotic EO cells |
| Formulation | 59.1% | 39.6% | 98.7% |
| Vehicle | | | |
| ApoA-IV | 29.3% | 63.1% | 92.4% |
| (3 mg) | | | |
| 1a | 42.5% | 53.2% | 95.7% |
| 1b | 41.8% | 55.0% | 96.8% |
| 3a | 51.6% | 43.7% | 95.3% |
| 5a | 42.9% | 53.1% | 96.0% |
| 6a | 54.5% | 42.8% | 97.3% |
| 7a | 59.4% | 39.9% | 99.3% |
| 8a | 31.2% | 60.1% | 95.3% |
| 9a | 39.6% | 56.4% | 96.0% |
| 10a | 58.2% | 40.3% | 98.5% |
| 11a | 49.7% | 44.4% | 94.1% |
| 12a | 52.0% | 42.1% | 94.1% |
| 13a | 51.3% | 44.0% | 95.3% |
| 14a | 26.6% | 67.1% | 95.7% |
| 15a | 38.6% | 57.9% | 96.5% |
| 16a | 50.2% | 44.3% | 94.5% |
| 17a | 53.5% | 48.2% | 98.7% |
| 18a | 41.0% | 55.5% | 96.5% |
| 19a | 52.7% | 41.8% | 94.5% |
| 20a | 59.3% | 39.4% | 98.7% |
| 21a | 38.2% | 58.0% | 96.2% |
| 22a | 28.1% | 68.7% | 96.8% |
| 23a | 31.4% | 65.6% | 97.0% |
| 24a | 33.8% | 64.9% | 98.7% |
| 25a | 22.3% | 73.1% | 95.4% |
| 26a | 30.0% | 67.3% | 97.3% |
| 27a | 39.1% | 56.5% | 95.6% |

### Example 19

### CD11b Change (100% as Baseline) on PMNL Surface Stimulated by 2.5 nm CCL11

(PMNL) cells were pretreated with 3 μg/mL ApoA-IV (positive control), 1 μg/mL test compounds, and formulation vehicle (negative control) for 30 minutes and incubated with serial dilutions of CCL 11 for 30 minutes at 37° C. Samples were stained with anti-CD16-PE-Cy5 and anti-CD11b-PE (ICRF44) antibodies. Eosinophils were identified as CD16 negative cells. CD11b upregulation was analyzed by flow cytometry and reported above. (Standard deviation in the assay is +/−2%). See results in Table 2 below.

TABLE 2

| Compound | CD11b change |
|---|---|
| No. | (100% as baseline) |
| Formulation | 147% |
| Vehicle | |
| ApoA-IV | 121% |
| (3 mg) | |
| 1a | 129% |
| 1b | 128% |
| 3a | 139% |
| 5a | 129% |
| 6a | 141% |
| 7a | 149% |
| 8a | 122% |
| 9a | 127% |
| 10a | 148% |
| 11a | 135% |
| 12a | 141% |

1. A lipopeptide, comprising:
alkenyl-[N-methyl aminoacid with hydrophobic side chain]-[aminoacid with polar uncharged sidechain]-[N-methyl aminoacid with hydrophobic side chain]-N-[(4-Hydroxy-2-pyrrolidinyl)carbonyl]-5-methyl-3-pyrrolin-2-one, or
alkenyl-[N-methyl aminoacid with hydrophobic side chain]-[aminoacid with polar uncharged sidechain]-[N-methyl aminoacid with hydrophobic side chain]-N-[4-hydroxypyrrolidine-2-carbonyl]-1H-[pyrrole-2,5-dione], or
alkenyl-[N-methyl aminoacid with hydrophobic side chain]-[aminoacid with polar uncharged sidechain]-[N-methyl aminoacid with hydrophobic side chain]-N-[pyrrolidine-2-carbonyl]-1H-[pyrrole-2,5-dione],
or a salt thereof, including any isomers of the foregoing.

2. A lipopeptide of formula (IV): 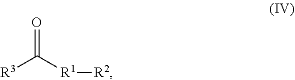
or a salt thereof, including any isomers of the foregoing, wherein:
R¹ is at least one optionally substituted amino acid moiety;
R² is 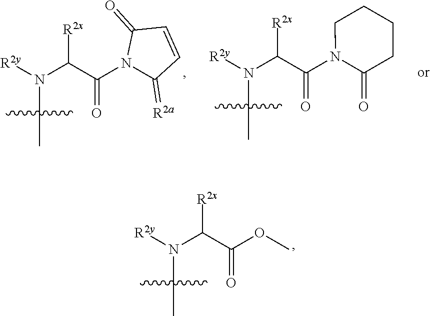
wherein
R^{2a} is H, oxo or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle; and
R³ is alkenyl.

3. The lipopeptide of claim 2, or a salt thereof, including any isomers of the foregoing, wherein R¹ is a sequence of three optionally substituted amino acid moieties.

4. The lipopeptide of claim 2, or a salt thereof, including any isomers of the foregoing, wherein R¹ is —R^{1a}—R^{1b}—R^{1c}—, wherein:
R^{1a} is an optionally substituted amino acid moiety with a hydrophobic side chain;
R^{1b} is an optionally substituted amino acid moiety with a polar uncharged side chain; and
R^{1c} is an optionally substituted amino acid moiety with a hydrophobic side chain.

5. The lipopeptide of claim 2, or a salt thereof, including any isomers of the foregoing, wherein R¹ is —R^{1a}—R^{1b}—R^{1c}, wherein:
R^{1a} is an optionally substituted Leu or Phe;
R^{1b} is optionally substituted Thr; and
R^{1c} is is optionally substituted Val.

6. The lipopeptide of claim claim 2, wherein R¹ is:
Leu-Thr-Val, wherein each Leu and Val is N-methylated;
Leu-OAcThr-Val, wherein each Leu and Val is N-methylated;
Phe-OAcThr-Val, wherein each Phe and Val is N-methylated; or
Phe-Thr-Val, wherein each Phe and Val is N-methylated.

7. The lipopeptide of claim 2, or a salt thereof, including any isomers of the foregoing, wherein:
R² is

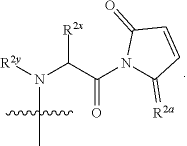

8. The lipopeptide of claim 7, or a salt thereof, including any isomers of the foregoing, wherein:
R^{2x} and R^{2y} are taken together with the atoms to which they are attached to form an optionally substituted 5-membered heterocycle.

9. The lipopeptide of claim 2, or a salt thereof, including any isomers of the foregoing, wherein:
R² is

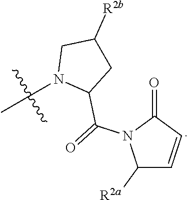

10. The lipopeptide of claim 9, or a salt thereof, including any isomers of the foregoing, wherein:
R^{2b} is H or OH.

11. The lipopeptide of claim 2, or a salt thereof, including any isomers of the foregoing, wherein:
R^{2a} is alkyl or oxo.

12. The lipopeptide of claim 2, or a salt thereof, including any isomers of the foregoing, wherein:
R³ is C₂-C₃₀ alkenyl.

13. A lipopeptide of formula (IV-A): 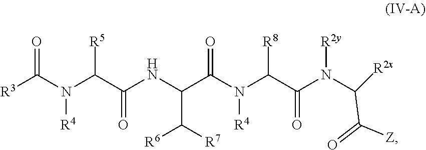
or a salt thereof, including any isomers of the foregoing, wherein:
Z is 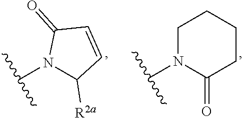
or alkoxy, wherein
R^{2a} is H, oxo or optionally substituted alkyl;
R^{2x} is alkyl, optionally substituted with —OH or —COOH;
R^{2y} is H or alkyl; or
R^{2x} and R^{2y} are taken together with the atoms to which they are attached to form an optionally substituted 5-membered heterocycle;
R³ is alkenyl;
R⁴ is H or alkyl;
each R⁵ and R⁸ is independently a hydrophobic side chain of Ala, Val, Ile, Leu, Met, Phe, Tyr, Trp, or Gly, wherein the hydrophobic side chain is optionally substituted;
R⁶ is H or alkyl; and
R⁷ is —OH, —O(C═O)alkyl, —SH, or —S(C═O)alkyl.

14. The lipopeptide of claim 13, or a salt thereof, including any isomers of the foregoing, wherein:
Z is

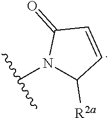

15. The lipopeptide of claim 14, or a salt thereof, including any isomers of the foregoing, wherein:
R^{2a} is alkyl or oxo.

16. The lipopeptide of claim 13, or a salt thereof, including any isomers of the foregoing, wherein:
R^{2x} and R^{2y} are taken together with the atoms to which they are attached to form 5-membered heterocycle substituted with —OH.

17. The lipopeptide of claim 13, or a salt thereof, including any isomers of the foregoing, wherein:
R³ is C₂-C₃₀ alkenyl.

18. The lipopeptide of claim 13, or a salt thereof, including any isomers of the foregoing, wherein:
R⁵ is the hydrophobic side chain of Leu or Phe.

19. The lipopeptide of claim 13, or a salt thereof, including any isomers of the foregoing, wherein:
R⁷ is —OH, —O(C═O)CH₃, —SH, or —S(C═O)CH₃.

20. A lipopeptide, wherein the lipopeptide is: 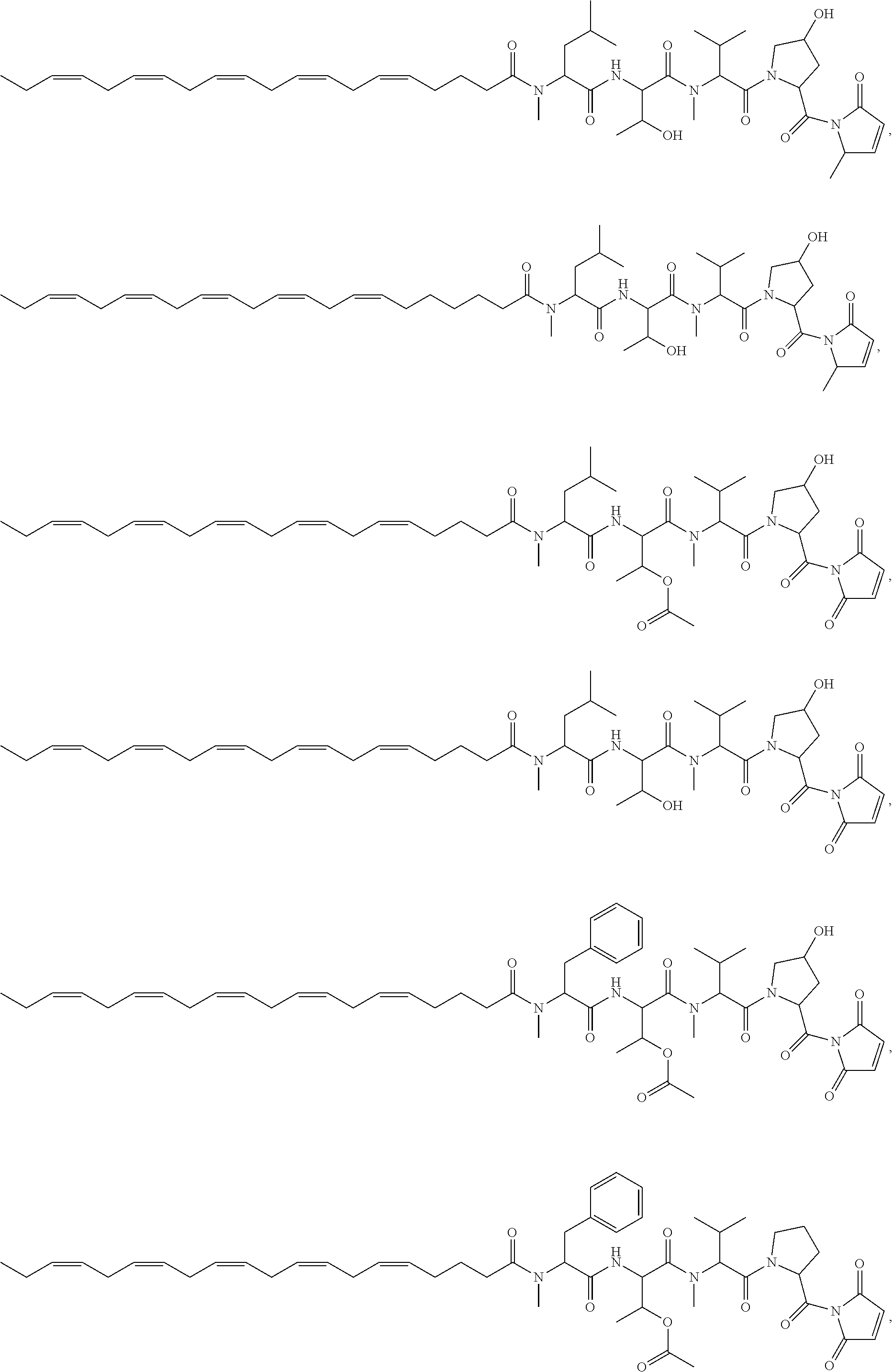 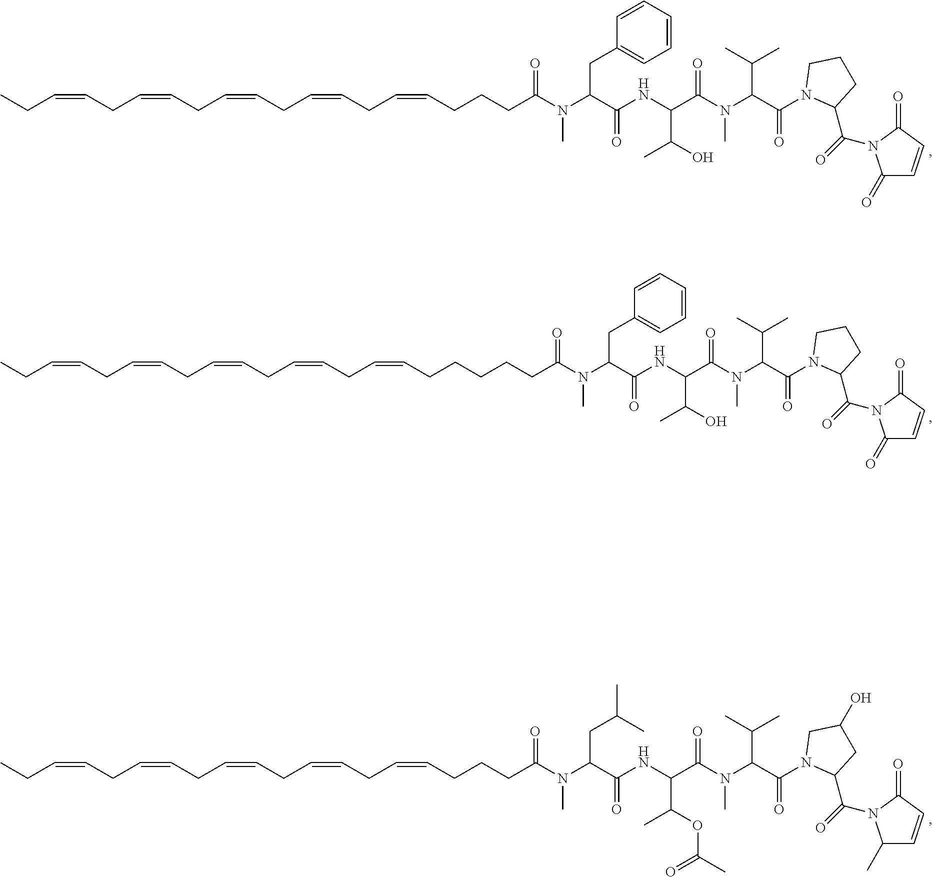 [SMILES: CC/C=C\C/C=C\C/C=C\C/C=C\C/C=C\CCCC(=O)N(C)C(Cc1ccccc1)C(=O)NC(C(=O)N(C)C(C(=O)N1CCCC1C(=O)N1C(=O)C=CC1=O)C(C)C)C(C)O ; CC/C=C\C/C=C\C/C=C\C/C=C\C/C=C\CCCCCC(=O)N(C)C(Cc1ccccc1)C(=O)NC(C(=O)N(C)C(C(=O)N1CCCC1C(=O)N1C(=O)C=CC1=O)C(C)C)C(C)O ; CC/C=C\C/C=C\C/C=C\C/C=C\C/C=C\CCCC(=O)N(C)C(CC(C)C)C(=O)NC(C(=O)N(C)C(C(=O)N1CC(O)CC1C(=O)N1C(=O)C=CC1C)C(C)C)C(C)OC(C)=O]
or a salt thereof, including any isomers of the foregoing.

21. The lipopeptide of claim 20, wherein the lipopeptide is: 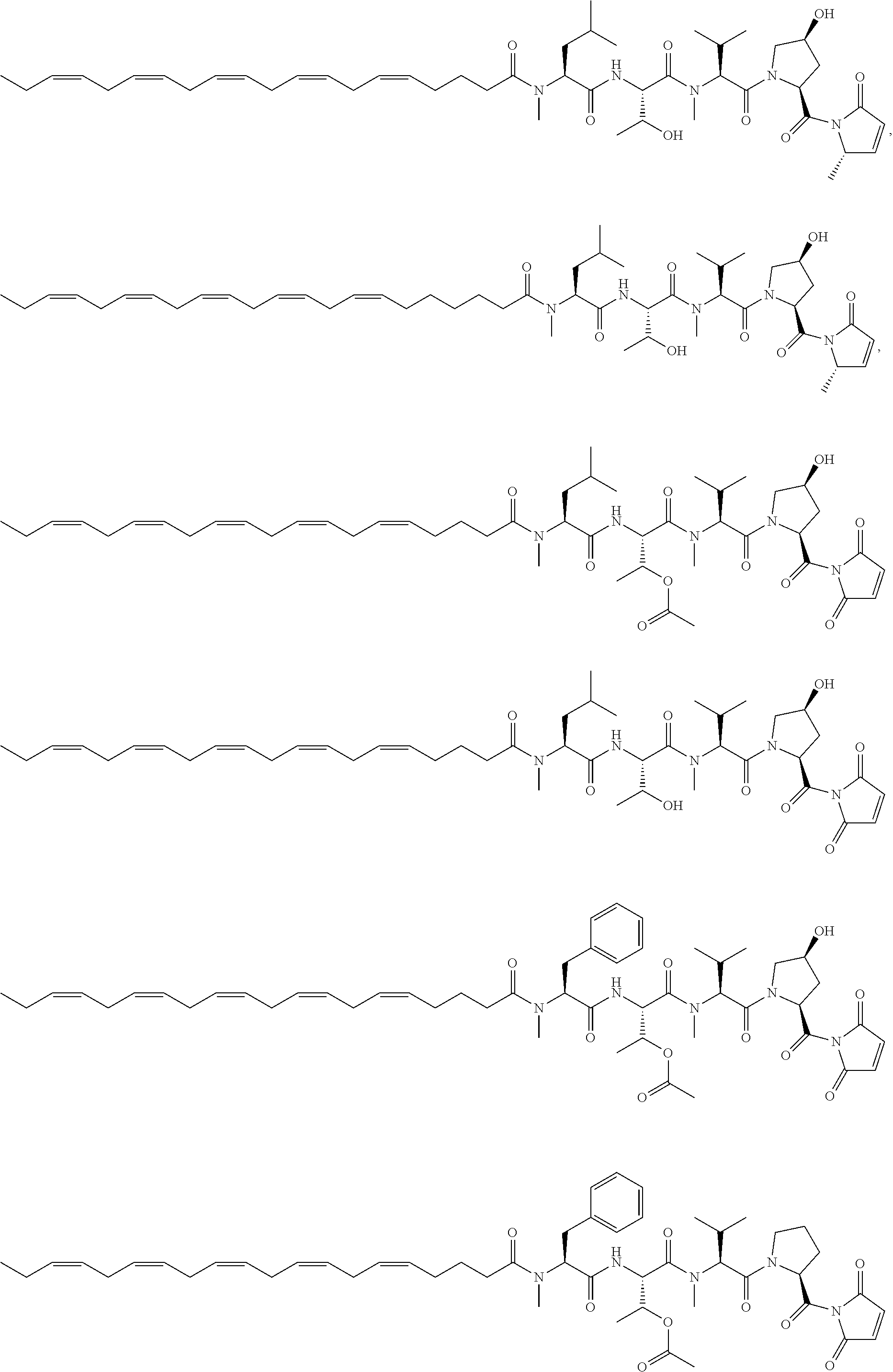 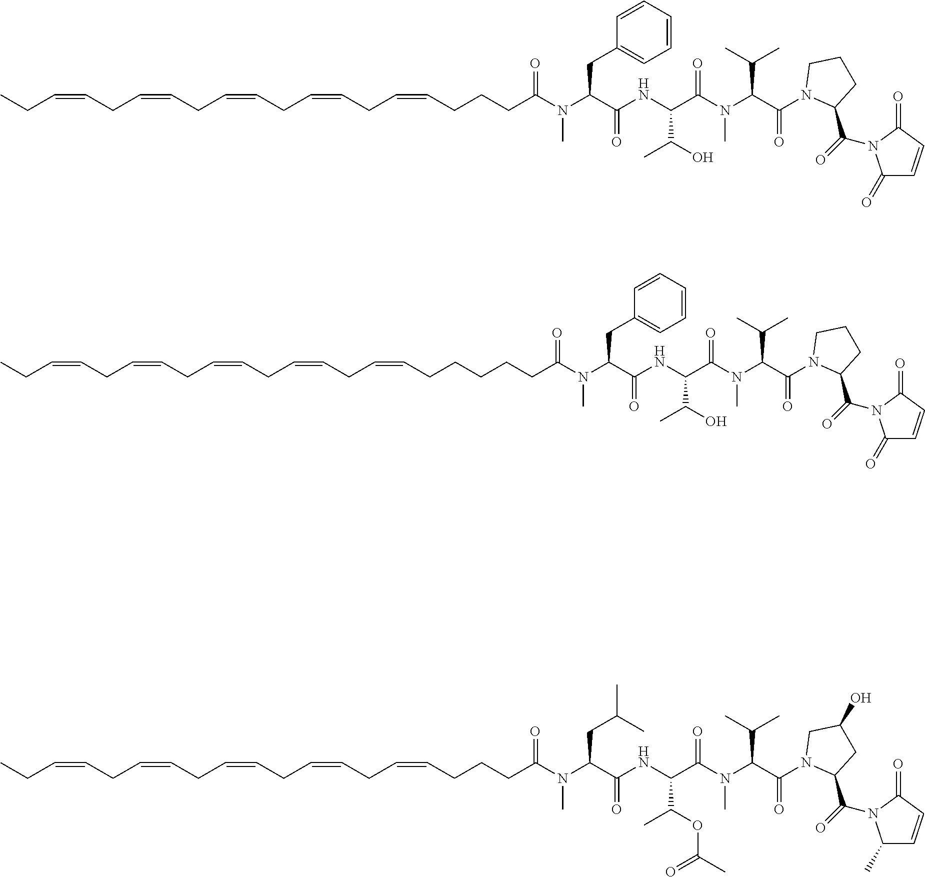 [SMILES: CC/C=C\C/C=C\C/C=C\C/C=C\C/C=C\CCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)O ; CC/C=C\C/C=C\C/C=C\C/C=C\C/C=C\CCCCCC(=O)N(C)[C@@H](Cc1ccccc1)C(=O)N[C@H](C(=O)N(C)[C@H](C(=O)N1CCC[C@H]1C(=O)N1C(=O)C=CC1=O)C(C)C)C(C)O ; CC/C=C\C/C=C\C/C=C\C/C=C\C/C=C\CCCC(=O)N(C)[C@@H](CC(C)C)C(=O)N[C@H](C(=O)N(C)[C@H](C(=O)N1C[C@@H](O)C[C@H]1C(=O)N1C(=O)C=C[C@@H]1C)C(C)C)C(C)OC(C)=O]
or a salt thereof, including any isomers of the foregoing.

22. A lipopeptide, wherein the lipopeptide is: 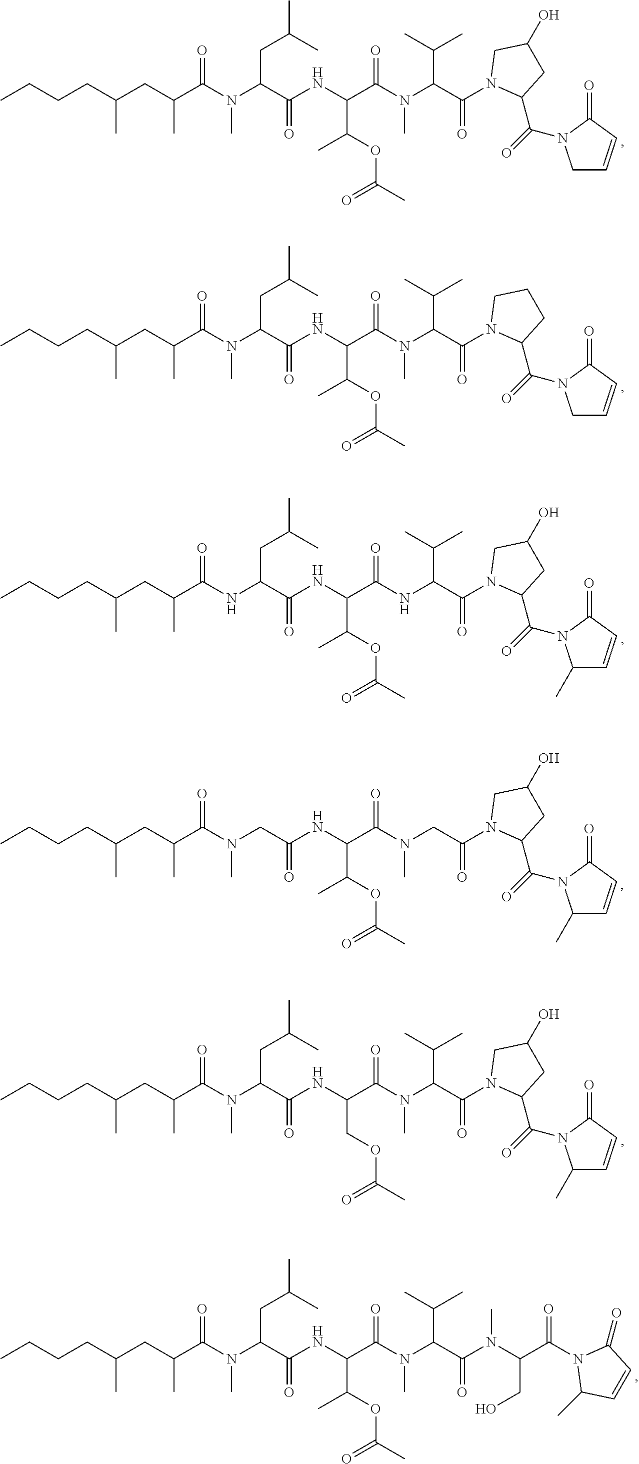 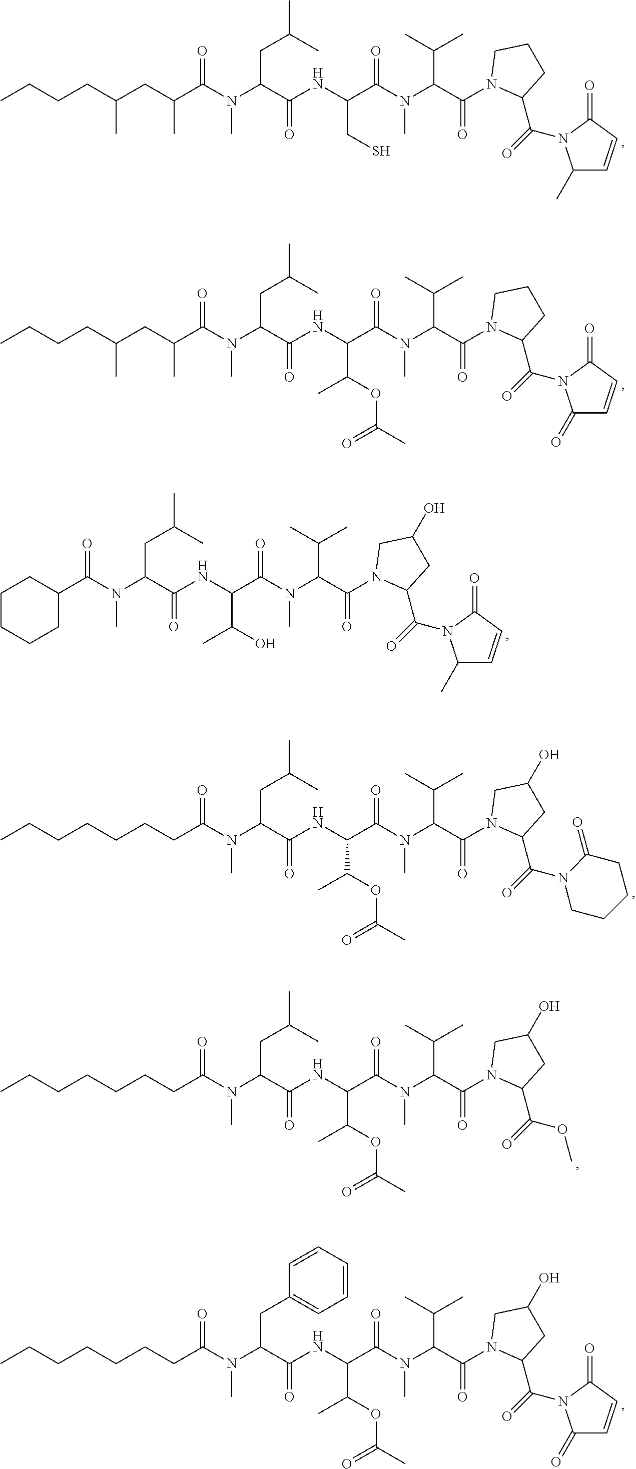 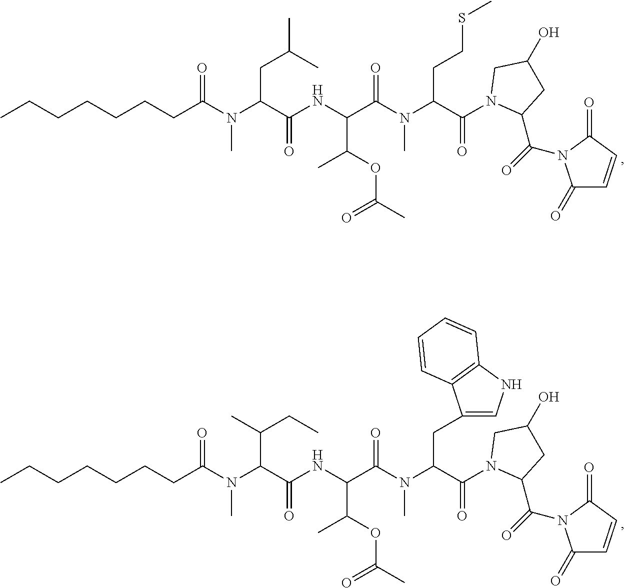 [SMILES: CCCCCCCC(=O)N(C)C(CC(C)C)C(=O)NC(C(=O)N(C)C(CCSC)C(=O)N1CC(O)CC1C(=O)N1C(=O)C=CC1=O)C(C)OC(C)=O ; CCCCCCCC(=O)N(C)C(C(=O)NC(C(=O)N(C)C(Cc1c[nH]c2ccccc12)C(=O)N1CC(O)CC1C(=O)N1C(=O)C=CC1=O)C(C)OC(C)=O)C(C)CC]
or a salt thereof, including any isomers of the foregoing.

23. The lipopeptide of claim 22, wherein the lipopeptide is: 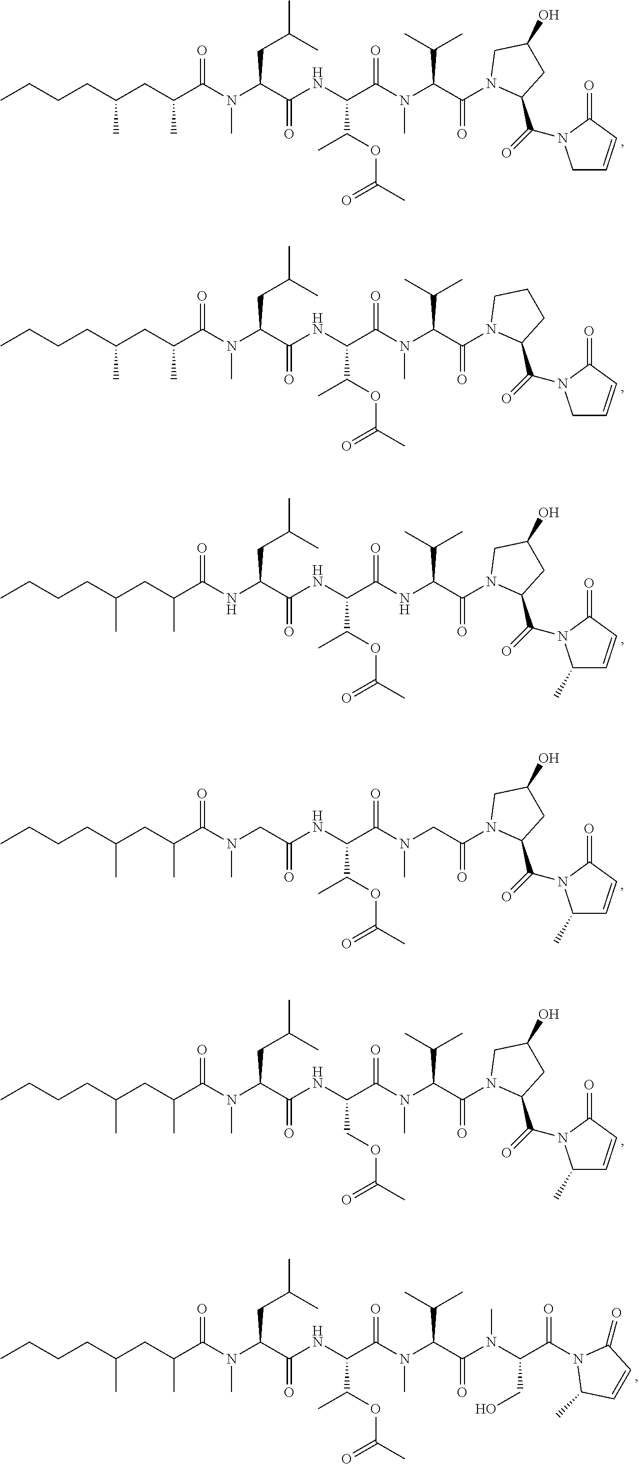 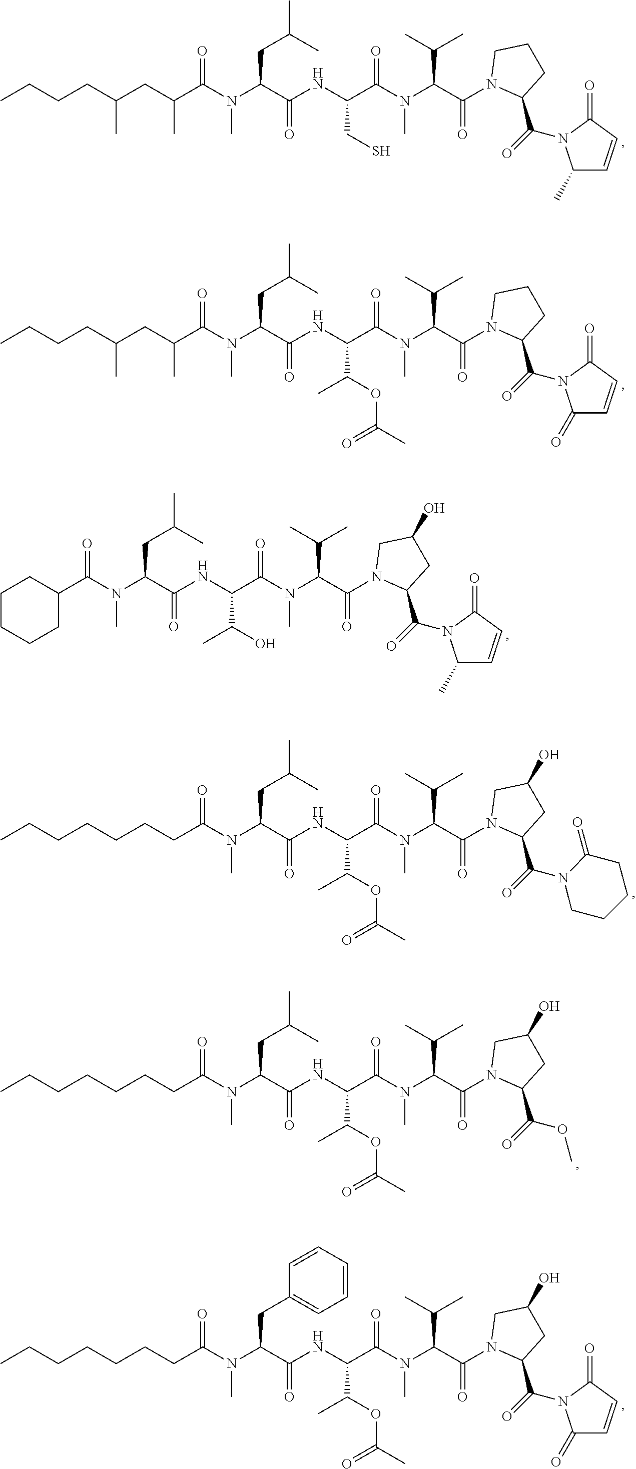 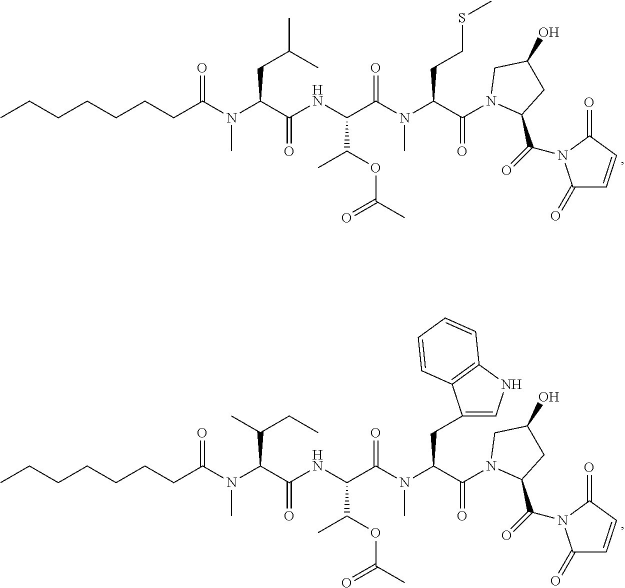 [SMILES: CCCCCCCC(=O)N(C)[C@@H](CC(C)C)C(=O)N[C@H](C(=O)N(C)[C@@H](CCSC)C(=O)N1C[C@@H](O)C[C@H]1C(=O)N1C(=O)C=CC1=O)C(C)OC(C)=O ; CCCCCCCC(=O)N(C)[C@H](C(=O)N[C@H](C(=O)N(C)[C@@H](Cc1c[nH]c2ccccc12)C(=O)N1C[C@@H](O)C[C@H]1C(=O)N1C(=O)C=CC1=O)C(C)OC(C)=O)C(C)CC]
or a salt thereof, including any isomers of the foregoing.

24. A pharmaceutical composition, comprising at least one lipopeptide of claim 1, and at least one pharmaceutically acceptable excipient.

25. A method for treating an eosinophilic inflammatory condition, disorder or disease in a human in need thereof, comprising: administering to the human an effective dose of a composition comprising at least one lipopeptide of claim 1 to treat the eosinophilic inflammatory condition, disorder or disease.

26. The method of claim 25, wherein the eosinophilic inflammatory condition, disorder or disease is a chronic inflammatory disorder of the airways.

27. The method of claim 25, wherein the eosinophilic inflammatory condition, disorder or disease is asthma.

28. The method of claim 25, wherein the eosinophilic inflammatory condition, disorder or disease is bronchial asthma.

29. A method for treating an eosinophilic respiratory condition, disorder or disease in a human in need thereof, comprising: administering to the human an effective dose of a composition comprising at least one lipopeptide of claim 1 to treat the eosinophilic respiratory condition, disorder or disease.

30. The method of claim 29, wherein the eosinophilic respiratory condition, disorder or disease is a viral respiratory disease.

31. The method of claim 29, wherein the eosinophilic respiratory condition, disorder or disease is severe acute respiratory syndrome.

32. The method of claim 29, wherein the severe acute respiratory syndrome is caused by a coronavirus.

33. The method of claim 25, wherein the human is largely resistant to medical and surgical interventions for treating the condition, disorder or disease.

34. The method of claim 25, wherein the human exhibits or has resistance to steroid treatments.

35. The method of claim 25, wherein the human has steroid treatment resistant asthma.

36. The method of claim 25, wherein the administration of the composition to the human reduces or delays the need to provide the human with assisted respiration.

37. A method for reducing eosinophil effector function in a human in need thereof, comprising administering to the human a salmonid oil composition or a composition comprising at least one lipopeptide of claim 1 to reduce eosinophil effector function.

38. (canceled)

39. (canceled)

40. (canceled)

41. The method of claim 25, wherein the composition is administered as a syrup, chewable, capsule or soft gel, or the composition is formulated for aerosol administration.

42. An article of manufacture, comprising:
a container comprising a composition comprising at least one lipopeptide of claim 1; and
a label containing instructions for use of such composition.

43. (canceled)

44. (canceled)

45. (canceled)

46. A kit, comprising:
a dosage form of a composition comprising a composition comprising at least one lipopeptide of claim 1; and
a package insert containing instructions for use of such composition.

47. (canceled)
